SEQUENCE LISTING (32 sequences)

SEQ ID NO: 1 - DNA, 951 bp, Escherichia coli
  source 1..951: mol_type = unassigned DNA
  misc_feature 1..951: note = CDS
atgagtaagcgtaaagtcgccattatcggttctggcaacattggtaccgatctgatgattaaaattttgcgtcacggtcagcatctggagatggcggtgatggttggcattgatcctcagtccgacggtctggcgcgcgccagacgtatgggcgtcgccaccacccatgaaggggtgatcggactgatgaacatgcctgaatttgctgatatcgacattgtatttgatgcgaccagcgccggtgctcatgtgaaaaacgatgccgctttacgcgaagcgaaaccggatattcgcttaattgacctgacgcctgctgccatcggcccttactgcgtgccggtggttaacctcgaggcgaacgtcgatcaactgaacgtcaacatggtcacctgcggcggccaggccaccattccaatggtggcggcagtttcacgcgtggcgcgtgttcattacgccgaaattatcgcttctatcgccagtaaatctgccggacctggcacgcgtgccaatatcgatgaatttacggaaaccacttcccgagccattgaagtggtgggcggcgcggcaaaagggaaggcgattattgtgcttaacccagcagagccaccgttgatgatgcgtgacacggtgtatgtattgagcgacgaagcttcacaagatgatatcgaagcctcaatcaatgaaatggctgaggcggtgcaggcttacgtaccgggttatcgcctgaaacagcgcgtgcagtttgaagttatcccgcaggataaaccggtcaatttaccgggcgtggggcaattctccggactgaaaacagcggtctggctggaagtcgaaggcgcagcgcattatctgcctgcctatgcgggcaacctcgacattatgacttccagtgcgctggcgacagcggaaaaaatggcccagtcactggcgcgcaaggcaggagaagcggcatga

SEQ ID NO: 2 - protein, 316 aa, Escherichia coli
MSKRKVAIIGSGNIGTDLMIKILRHGQHLEMAVMVGIDPQSDGLARARRMGVATTHEGVIGLMNMPEFADIDIVFDATSAGAHVKNDAALREAKPDIRLIDLTPAAIGPYCVPVVNLEANVDQLNVNMVTCGGQATIPMVAAVSRVARVHYAEIIASIASKSAGPGTRANIDEFTETTSRAIEVVGGAAKGKAIIVLNPAEPPLMMRDTVYVLSDEASQDDIEASINEMAEAVQAYVPGYRLKQRVQFEVIPQDKPVNLPGVGQFSGLKTAVWLEVEGAAHYLPAYAGNLDIMTSSALATAEKMAQSLARKAGEAA

SEQ ID NO: 3 - DNA, 58 bp, synthetic construct
  misc_feature 1..58: note = primer
  source 1..58: mol_type = other DNA
ggggacaagtttgtacaaaaaagcaggctatgagtaagcgtaaagtcgccattatcgg

SEQ ID NO: 4 - DNA, 54 bp, synthetic construct
  misc_feature 1..54: note = primer
  source 1..54: mol_type = other DNA
ggggaccactttgtacaagaaagctgggtgttcatgccgcttctcctgccttgc

SEQ ID NO: 5 - DNA, 64 bp, synthetic construct
  misc_feature 1..64: note = primer
  source 1..64: mol_type = other DNA
ttgtacaccccccccctccacaaacacaaatattgataatataaacagctgaagcttcgtacgc

SEQ ID NO: 6 - DNA, 66 bp, synthetic construct
  misc_feature 1..66: note = primer
  source 1..66: mol_type = other DNA
aatctaatcttcatgtagatctaattcttcaatcatgtccggcggcataggccactagtggatctg

SEQ ID NO: 7 - DNA, 65 bp, synthetic construct
  misc_feature 1..65: note = primer
  source 1..65: mol_type = other DNA
tcaattctctttccctttccttttccttcgctccccttccttatcccaggctgaagcttcgtacg

SEQ ID NO: 8 - DNA, 66 bp, synthetic construct
  misc_feature 1..66: note = primer
  source 1..66: mol_type = other DNA
gtgtctattcgtcatcgatgtctagctcttcaatcatctccggtaggcataggccactagtggatc

SEQ ID NO: 9 - DNA, 20 bp, synthetic construct
  misc_feature 1..20: note = primer
  source 1..20: mol_type = other DNA
cccacccacaccaccaatac

SEQ ID NO: 10 - DNA, 20 bp, synthetic construct
  misc_feature 1..20: note = primer
  source 1..20: mol_type = other DNA
cggacgccagatgctagaag

SEQ ID NO: 11 - DNA, 20 bp, synthetic construct
  misc_feature 1..20: note = primer
  source 1..20: mol_type = other DNA
gttcagcagctcttctctac

SEQ ID NO: 12 - DNA, 20 bp, synthetic construct
  misc_feature 1..20: note = primer
  source 1..20: mol_type = other DNA
ccaaatgcgacatgagtcac

SEQ ID NO: 13 - DNA, 20 bp, synthetic construct
  misc_feature 1..20: note = primer
  source 1..20: mol_type = other DNA
cgcacgtcaagactgtcaag

SEQ ID NO: 14 - DNA, 20 bp, synthetic construct
  misc_feature 1..20: note = primer
  source 1..20: mol_type = other DNA
tcgtatgtgaatgctggtcg

SEQ ID NO: 15 - DNA, 20 bp, synthetic construct
  misc_feature 1..20: note = primer
  source 1..20: mol_type = other DNA
cgcacgtcaagactgtcaag

SEQ ID NO: 16 - DNA, 20 bp, synthetic construct
  misc_feature 1..20: note = primer
  source 1..20: mol_type = other DNA
tcgtatgtgaatgctggtcg

SEQ ID NO: 17 - DNA, 2142 bp, Saccharomyces cerevisiae
  source 1..2142: mol_type = unassigned DNA
atgtcgccctctgccgtacaatcatcaaaactagaagaacagtcaagtgaaattgacaagttgaaagcaaaaatgtcccagtctgccgccactgcgcagcagaagaaggaacatgagtatgaacatttgacttcggtcaagatcgtgccacaacggcccatctcagatagactgcagcccgcaattgctacccactattctccacacttggacgggttgcaggactatcagcgcttgcacaaggagtctattgaagaccctgctaagttcttcggttctaaagctacccaatttttaaactggtctaagccattcgataaggtgttcatcccagaccctaaaacgggcaggccctccttccagaacaatgcatggttcctcaacggccaattaaacgcctgttacaactgtgttgacagacatgccttgaagactcctaacaagaaagccattattttcgaaggtgacgagcctggccaaggctattccattacctacaaggaactacttgaagaagtttgtcaagtggcacaagtgctgacttactctatgggcgttcgcaagggcgatactgttgccgtgtacatgcctatggtcccagaagcaatcataaccttgttggccatttcccgtatcggtgccattcactccgtagtctttgccgggttttcttccaactccttgagagatcgtatcaacgatggggactctaaagttgtcatcactacagatgaatccaacagaggtggtaaagtcattgagactaaaagaattgttgatgacgcgctaagagagaccccaggcgtgagacacgtcttggtttatagaaagaccaacaatccatctgttgctttccatgcccccagagatttggattgggcaacagaaaagaagaaatacaagacctactatccatgcacacccgttgattctgaggatccattattcttgttgtatacgtctggttctactggtgcccccaagggtgttcaacattctaccgcaggttacttgctgggagctttgttgaccatgcgctacacttttgacactcaccaagaagacgttttcttcacagctggagacattggctggattacaggccacacttatgtggtttatggtcccttactatatggttgtgccactttggtctttgaagggactcctgcgtacccaaattactcccgttattgggatattattgatgaacacaaagtcacccaattttatgttgcgccaactgctttgcgtttgttgaaaagagctggtgattcctacatcgaaaatcattccttaaaatctttgcgttgcttgggttcggtcggtgagccaattgctgctgaagtttgggagtggtactctgaaaaaataggtaaaaatgaaatccccattgtagacacctactggcaaacagaatctggttcgcatctggtcaccccgctggctggtggtgttacaccaatgaaaccgggttctgcctcattccccttcttcggtattgatgcagttgttcttgaccctaacactggtgaagaacttaacaccagccacgcagagggtgtccttgccgtcaaagctgcatggccatcatttgcaagaactatttggaaaaatcatgataggtatctagacacttatttgaacccttaccctggctactatttcactggtgatggtgctgcaaaggataaggatggttatatctggattttgggtcgtgtagacgatgtggtgaacgtctctggtcaccgtctgtctaccgctgaaattgaggctgctattatcgaagatccaattgtggccgagtgtgctgttgtcggattcaacgatgacttgactggtcaagcagttgctgcatttgtggtgttgaaaaacaaatctagttggtccaccgcaacagatgatgaattacaagatatcaagaagcatttggtctttactgttagaaaagacatcgggccatttgccgcaccaaaattgatcattttagtggatgacttgcccaagacaagatccggcaaaattatgagacgtattttaagaaaaatcctagcaggagaaagtgaccaactaggcgacgtttctacattgtcaaaccctggcattgttagacatctaattgattcggtcaagttgtaa

SEQ ID NO: 18 - DNA, 2052 bp, Saccharomyces cerevisiae
  source 1..2052: mol_type = unassigned DNA
atgacaatcaaggaacataaagtagtttatgaagctcacaacgtaaaggctcttaaggctcctcaacatttttacaacagccaacccggcaagggttacgttactgatatgcaacattatcaagaaatgtatcaacaatctatcaatgagccagaaaaattctttgataagatggctaaggaatacttgcattgggatgctccatacaccaaagttcaatctggttcattgaacaatggtgatgttgcatggtttttgaacggtaaattgaatgcatcatacaattgtgttgacagacatgcctttgctaatcccgacaagccagctttgatctatgaagctgatgacgaatccgacaacaaaatcatcacatttggtgaattactcagaaaagtttcccaaatcgctggtgtcttaaaaagctggggcgttaagaaaggtgacacagtggctatctatttgccaatgattccagaagcggtcattgctatgttggctgtggctcgtattggtgctattcactctgttgtctttgctgggttctccgctggttcgttgaaagatcgtgtcgttgacgctaattctaaagtggtcatcacttgtgatgaaggtaaaagaggtggtaagaccatcaacactaaaaaaattgttgacgaaggtttgaacggagtcgatttggtttcccgtatcttggttttccaaagaactggtactgaaggtattccaatgaaggccggtagagattactggtggcatgaggaggccgctaagcagagaacttacctacctcctgtttcatgtgacgctgaagatcctctatttttattatacacttccggttccactggttctccaaagggtgtcgttcacactacaggtggttatttattaggtgccgctttaacaactagatacgtttttgatattcacccagaagatgttctcttcactgccggtgacgtcggctggatcacgggtcacacctatgctctatatggtccattaaccttgggtaccgcctcaataattttcgaatccactcctgcctacccagattatggtagatattggagaattatccaacgtcacaaggctacccatttctatgtggctccaactgctttaagattaatcaaacgtgtaggtgaagccgaaattgccaaatatgacacttcctcattacgtgtcttgggttccgtcggtgaaccaatctctccagacttatgggaatggtatcatgaaaaagtgggtaacaaaaactgtgtcatttgtgacactatgtggcaaacagagtctggttctcatttaattgctcctttggcaggtgctgtcccaacaaaacctggttctgctaccgtgccattctttggtattaacgcttgtatcattgaccctgttacaggtgtggaattagaaggtaatgatgtcgaaggtgtccttgccgttaaatcaccatggccatcaatggctagatctgtttggaaccaccacgaccgttacatggatacttacttgaaaccttatcctggtcactatttcacaggtgatggtgctggtagagatcatgatggttactactggatcaggggtagagttgacgacgttgtaaatgtttccggtcatagattatccacatcagaaattgaagcatctatctcaaatcacgaaaacgtctcggaagctgctgttgtcggtattccagatgaattgaccggtcaaaccgtcgttgcatatgtttccctaaaagatggttatctacaaaacaacgctactgaaggtgatgcagaacacatcacaccagataatttacgtagagaattgatcttacaagttaggggtgagattggtcctttcgcctcaccaaaaaccattattctagttagagatctaccaagaacaaggtcaggaaagattatgagaagagttctaagaaaggttgcttctaacgaagccgaacagctaggtgacctaactactttggccaacccagaagttgtacctgccatcatttctgctgtagagaaccaatttttctctcaaaaaaagaaataa

SEQ ID NO: 19 - DNA, 1047 bp, Saccharomyces cerevisiae
  source 1..1047: mol_type = unassigned DNA
atgtctatcccagaaactcaaaaaggtgttatcttctacgaatcccacggtaagttggaatacaaagatattccagttccaaagccaaaggccaacgaattgttgatcaacgttaaatactctggtgtctgtcacactgacttgcacgcttggcacggtgactggccattgccagttaagctaccattagtcggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaacaatacgctaccgctgacgctgttcaagccgctcacattcctcaaggtaccgacttggcccaagtcgcccccatcttgtgtgctggtatcaccgtctacaaggctttgaagtctgctaacttgatggccggtcactgggttgctatctccggtgctgctggtggtctaggttctttggctgttcaatacgccaaggctatgggttacagagtcttgggtattgacggtggtgaaggtaaggaagaattattcagatccatcggtggtgaagtcttcattgacttcactaaggaaaaggacattgtcggtgctgttctaaaggccactgacggtggtgctcacggtgtcatcaacgtttccgtttccgaagccgctattgaagcttctaccagatacgttagagctaacggtaccaccgttttggtcggtatgccagctggtgccaagtgttgttctgatgtcttcaaccaagtcgtcaagtccatctctattgttggttcttacgtcggtaacagagctgacaccagagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataa

SEQ ID NO: 20 - DNA, 1047 bp, Saccharomyces cerevisiae
  source 1..1047: mol_type = unassigned DNA
atgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataa

SEQ ID NO: 21 - DNA, 1128 bp, Saccharomyces cerevisiae
  source 1..1128: mol_type = unassigned DNA
atgttgagaacgtcaacattgttcaccaggcgtgtccaaccaagcctattttctagaaacattcttagattgcaatccacagctgcaatccctaagactcaaaaaggtgtcatcttttatgagaataaggggaagctgcattacaaagatatccctgtccccgagcctaagccaaatgaaattttaatcaacgttaaatattctggtgtatgtcacaccgatttacatgcttggcacggcgattggccattacctgttaaactaccattagtaggtggtcatgaaggtgctggtgtagttgtcaaactaggttccaatgtcaagggctggaaagtcggtgatttagcaggtatcaaatggctgaacggttcttgtatgacatgcgaattctgtgaatcaggtcatgaatcaaattgtccagatgctgatttatctggttacactcatgatggttctttccaacaatttgcgaccgctgatgctattcaagccgccaaaattcaacagggtaccgacttggccgaagtagccccaatattatgtgctggtgttactgtatataaagcactaaaagaggcagacttgaaagctggtgactgggttgccatctctggtgctgcaggtggcttgggttccttggccgttcaatatgcaactgcgatgggttacagagttctaggtattgatgcaggtgaggaaaaggaaaaacttttcaagaaattggggggtgaagtattcatcgactttactaaaacaaagaatatggtttctgacattcaagaagctaccaaaggtggccctcatggtgtcattaacgtttccgtttctgaagccgctatttctctatctacggaatatgttagaccatgtggtaccgtcgttttggttggtttgcccgctaacgcctacgttaaatcagaggtattctctcatgtggtgaagtccatcaatatcaagggttcttatgttggtaacagagctgatacgagagaagccttagacttctttagcagaggtttgatcaaatcaccaatcaaaattgttggattatctgaattaccaaaggtttatgacttgatggaaaagggcaagattttgggtagatacgtcgtcgatactagtaaataa

SEQ ID NO: 22 - DNA, 1149 bp, Saccharomyces cerevisiae
  source 1..1149: mol_type = unassigned DNA
atgtcttccgttactgggttttacattccaccaatctctttctttggtgaaggtgctttagaagaaaccgctgattacatcaaaaacaaggattacaaaaaggctttgatcgttactgatcctggtattgcagctattggtctctccggtagagtccaaaagatgttggaagaacgtgacttaaacgttgctatctatgacaaaactcaaccaaacccaaatattgccaatgtcacagctggtttgaaggttttgaaggaacaaaactctgaaattgttgtttccattggtggtggttctgctcacgacaatgctaaggccattgctttattggctactaacggtggggaaatcggagactatgaaggtgtcaatcaatctaagaaggctgctttaccactatttgccatcaacactactgctggtactgcttccgaaatgaccagattcactattatctctaatgaagaaaagaaaatcaagatggctatcattgacaacaacgtcactccagctgttgctgtcaacgatccatctaccatgtttggtttgccacctgctttgactgctgctactggtctagatgctttgactcactgtatcgaagcttatgtttccaccgcctctaacccaatcaccgatgcctgtgctttgaagggtattgatttgatcaatgaaagcttagtcgctgcatacaaagacggtaaagacaagaaggccagaactgacatgtgttacgctgaatacttggcaggtatggctttcaacaatgcttctctaggttatgttcatgcccttgctcatcaacttggtggtttctaccacttgcctcatggtgtttgtaacgctgtcttgttgcctcatgttcaagaggccaacatgcaatgtccaaaggccaagaagagattaggtgaaattgctttgcatttcggtgcttctcaagaagatccagaagaaaccatcaaggctttgcacgttttaaacagaaccatgaacattccaagaaacttgaaagaattaggtgttaaaaccgaagattttgaaattttggctgaacacgccatgcatgatgcctgccatttgactaacccagttcaattcaccaaagaacaagtggttgccattatcaagaaagcctatgaatattaa

SEQ ID NO: 23 - DNA, 1056 bp, Saccharomyces cerevisiae
  source 1..1056: mol_type = unassigned DNA
atgccttcgcaagtcattcctgaaaaacaaaaggctattgtcttttatgagacagatggaaaattggaatataaagacgtcacagttccggaacctaagcctaacgaaattttagtccacgttaaatattctggtgtttgtcatagtgacttgcacgcgtggcacggtgattggccatttcaattgaaatttccattaatcggtggtcacgaaggtgctggtgttgttgttaagttgggatctaacgttaagggctggaaagtcggtgattttgcaggtataaaatggttgaatgggacttgcatgtcctgtgaatattgtgaagtaggtaatgaatctcaatgtccttatttggatggtactggcttcacacatgatggtacttttcaagaatacgcaactgccgatgccgttcaagctgcccatattccaccaaacgtcaatcttgctgaagttgccccaatcttgtgtgcaggtatcactgtttataaggcgttgaaaagagccaatgtgataccaggccaatgggtcactatatccggtgcatgcggtggcttgggttctctggcaatccaatacgcccttgctatgggttacagggtcattggtatcgatggtggtaatgccaagcgaaagttatttgaacaattaggcggagaaatattcatcgatttcacggaagaaaaagacattgttggtgctataataaaggccactaatggcggttctcatggagttattaatgtgtctgtttctgaagcagctatcgaggcttctacgaggtattgtaggcccaatggtactgtcgtcctggttggtatgccagctcatgcttactgcaattccgatgttttcaatcaagttgtaaaatcaatctccatcgttggatcttgtgttggaaatagagctgatacaagggaggctttagatttcttcgccagaggtttgatcaaatctccgatccacttagctggcctatcggatgttcctgaaatttttgcaaagatggagaagggtgaaattgttggtagatatgttgttgagacttctaaatga

SEQ ID NO: 24 - DNA, 1176 bp, Saccharomyces cerevisiae
  source 1..1176: mol_type = unassigned DNA
atgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattag

SEQ ID NO: 25 - DNA, 1323 bp, Saccharomyces cerevisiae
  source 1..1323: mol_type = unassigned DNA
atgcttgctgtcagaagattaacaagatacacattccttaagcgaacgcatccggtgttatatactcgtcgtgcatataaaattttgccttcaagatctactttcctaagaagatcattattacaaacacaactgcactcaaagatgactgctcatactaatatcaaacagcacaaacactgtcatgaggaccatcctatcagaagatcggactctgccgtgtcaattgtacatttgaaacgtgcgcccttcaaggttacagtgattggttctggtaactgggggaccaccatcgccaaagtcattgcggaaaacacagaattgcattcccatatcttcgagccagaggtgagaatgtgggtttttgatgaaaagatcggcgacgaaaatctgacggatatcataaatacaagacaccagaacgttaaatatctacccaatattgacctgccccataatctagtggccgatcctgatcttttacactccatcaagggtgctgacatccttgttttcaacatccctcatcaatttttaccaaacatagtcaaacaattgcaaggccacgtggcccctcatgtaagggccatctcgtgtctaaaagggttcgagttgggctccaagggtgtgcaattgctatcctcctatgttactgatgagttaggaatccaatgtggcgcactatctggtgcaaacttggcaccggaagtggccaaggagcattggtccgaaaccaccgtggcttaccaactaccaaaggattatcaaggtgatggcaaggatgtagatcataagattttgaaattgctgttccacagaccttacttccacgtcaatgtcatcgatgatgttgctggtatatccattgccggtgccttgaagaacgtcgtggcacttgcatgtggtttcgtagaaggtatgggatggggtaacaatgcctccgcagccattcaaaggctgggtttaggtgaaattatcaagttcggtagaatgtttttcccagaatccaaagtcgagacctactatcaagaatccgctggtgttgcagatctgatcaccacctgctcaggcggtagaaacgtcaaggttgccacatacatggccaagaccggtaagtcagccttggaagcagaaaaggaattgcttaacggtcaatccgcccaagggataatcacatgcagagaagttcacgagtggctacaaacatgtgagttgacccaagaattcccattattcgaggcagtctaccagatagtctacaacaacgtccgcatggaagacctaccggagatgattgaagagctagacatcgatgacgaatag

SEQ ID NO: 26 - DNA, 753 bp, Saccharomyces cerevisiae
  source 1..753: mol_type = unassigned DNA
atgcctttgaccacaaaacctttatctttgaaaatcaacgccgctctattcgatgttgacggtaccatcatcatctctcaaccagccattgctgctttctggagagatttcggtaaagacaagccttacttcgatgccgaacacgttattcacatctctcacggttggagaacttacgatgccattgccaagttcgctccagactttgctgatgaagaatacgttaacaagctagaaggtgaaatcccagaaaagtacggtgaacactccatcgaagttccaggtgctgtcaagttgtgtaatgctttgaacgccttgccaaaggaaaaatgggctgtcgccacctctggtacccgtgacatggccaagaaatggttcgacattttgaagatcaagagaccagaatacttcatcaccgccaatgatgtcaagcaaggtaagcctcacccagaaccatacttaaagggtagaaacggtttgggtttcccaattaatgaacaagacccatccaaatctaaggttgttgtctttgaagacgcaccagctggtattgctgctggtaaggctgctggctgtaaaatcgttggtattgctaccactttcgatttggacttcttgaaggaaaagggttgtgacatcattgtcaagaaccacgaatctatcagagtcggtgaatacaacgctgaaaccgatgaagtcgaattgatctttgatgactacttatacgctaaggatgacttgttgaaatggtaa

SEQ ID NO: 27 - DNA, 753 bp, Saccharomyces cerevisiae
  source 1..753: mol_type = unassigned DNA
atgggattgactactaaacctctatctttgaaagttaacgccgctttgttcgacgtcgacggtaccattatcatctctcaaccagccattgctgcattctggagggatttcggtaaggacaaaccttatttcgatgctgaacacgttatccaagtctcgcatggttggagaacgtttgatgccattgctaagttcgctccagactttgccaatgaagagtatgttaacaaattagaagctgaaattccggtcaagtacggtgaaaaatccattgaagtcccaggtgcagttaagctgtgcaacgctttgaacgctctaccaaaagagaaatgggctgtggcaacttccggtacccgtgatatggcacaaaaatggttcgagcatctgggaatcaggagaccaaagtacttcattaccgctaatgatgtcaaacagggtaagcctcatccagaaccatatctgaagggcaggaatggcttaggatatccgatcaatgagcaagacccttccaaatctaaggtagtagtatttgaagacgctccagcaggtattgccgccggaaaagccgccggttgtaagatcattggtattgccactactttcgacttggacttcctaaaggaaaaaggctgtgacatcattgtcaaaaaccacgaatccatcagagttggcggctacaatgccgaaacagacgaagttgaattcatttttgacgactacttatatgctaaggacgatctgttgaaatggtaa

SEQ ID NO: 28 - DNA, 939 bp, Saccharomyces cerevisiae
  source 1..939: mol_type = unassigned DNA
  misc_feature 1..939: note = CDS
atgaatcagaaactgaaagtagctatcataggttccggtaatatcggaacagacctgatgattaaggtactgcgtaatgcaaagtacttagaaatgggcgcgatggtcggtatcgatgcagcctctgatggactggccagagctcaaagaatgggcgttacgactacttatgcaggtgtagaagggctaatcaagcttcctgaatttgcagacatagatttcgtcttcgatgctacatctgcatcagcccacgttcagaacgaggctttattaagacaagctaaacctggtattagattgatcgaccttaccccggcggcaatcggtccttactgtgtccccgtagttaatctcgaggaacatttgggcaagttgaacgttaacatggttacttgcggtggccaagctactattccgatggtcgcagctgtctcacgtgtagccaaagtccattatgctgagattgttgcttctatttcaagcaagagtgccggacctggaaccagagccaatatagatgaattcactgagacaaccagtaaagccatagaagttattggtggtgctgcaaagggtaaagctataattattatgaacccagctgaaccaccattgattatgagggatacggtgtatgtgctttccgccgccgctgatcaagccgctgtcgcagcttctgtggctgaaatggttcaagcggttcaagcatacgtgccaggctataggttaaaacaacaggttcagtttgacgtgattcccgagtccgcgccactaaacatccccggtttggggagattcagcgggttgaaaacaagtgtgttcctagaagtagaaggtgctgctcattatttgccagcatacgcaggaaacttagatattatgacttccgcagcgttagctacagccgaacgtatggcgcaatcaatgttgaatgcatag

SEQ ID NO: 29 - protein, 312 aa, Saccharomyces cerevisiae
MNQKLKVAIIGSGNIGTDLMIKVLRNAKYLEMGAMVGIDAASDGLARAQRMGVTTTYAGVEGLIKLPEFADIDFVFDATSASAHVQNEALLRQAKPGIRLIDLTPAAIGPYCVPVVNLEEHLGKLNVNMVTCGGQATIPMVAAVSRVAKVHYAEIVASISSKSAGPGTRANIDEFTETTSKAIEVIGGAAKGKAIIIMNPAEPPLIMRDTVYVLSAAADQAAVAASVAEMVQAVQAYVPGYRLKQQVQFDVIPESAPLNIPGLGRFSGLKTSVFLEVEGAAHYLPAYAGNLDIMTSAALATAERMAQSMLNA

SEQ ID NO: 30 - DNA, 18 bp, synthetic construct
  misc_feature 1..18: note = primer
  source 1..18: mol_type = other DNA
cattgattgcgccatacg

SEQ ID NO: 31 - DNA, 20 bp, synthetic construct
  misc_feature 1..20: note = primer
  source 1..20: mol_type = other DNA
ccggtaatatcggaacagac

SEQ ID NO: 32 - DNA, 939 bp, Pseudomonas sp.
  source 1..939: mol_type = unassigned DNA
atgaaccagaaactcaaagtcgcgatcatcggttcgggcaatatcggcaccgacctgatgatcaaggtgctgcgcaacgccaagtacctggaaatgggcgccatggtcggcatcgacgccgcctccgacggcctggcccgcgcccagcgcatgggcgtgacgaccacctatgccggcgtcgaagggctgatcaagctgcccgaattcgccgacatcgatttcgtcttcgacgccacctcggccagtgcccacgtgcagaacgaggcgctgctgcgccaggccaaacctggcatccgcctgatcgacctgaccccggcggccatcggcccgtactgcgtgccggtggtcaatctggaggagcacctcggcaagctcaacgtcaacatggttacctgcggcggccaggcgaccatcccgatggtcgccgcggtctcccgtgtggccaaggtccattacgccgagatcgtcgcctcgatcagcagcaagtcggccggacccggcacccgcgccaacatcgacgagttcaccgagaccaccagcaaagccatcgaagtgatcggtggtgcggccaagggcaaggcgatcatcatcatgaacccggctgagccgccgctgatcatgcgcgacaccgtgtatgtgctgtccgccgccgccgatcaggccgccgtcgcggcctcggtggcggaaatggttcaggcggtgcaggcctacgtgcccggctatcgcctgaagcagcaggtgcagttcgacgtgatccccgagtccgcgccgctgaacatccccggtctcggccggttcagcgggttgaagacctcggtgttcctcgaagtcgaaggcgccgcccattacctgccggcctacgccggcaacctcgacatcatgacctccgccgcgctggctaccgccgagcgtatggcgcagtcgatgttgaacgcctga

(19) United States
(12) Patent Application Publication
(10) Pub. No.: US 2026/0009044 A1
(43) Pub. Date: Jan. 8, 2026

(54) FERMENTATIVE GLYCEROL-FREE ETHANOL PRODUCTION

(71) Applicant: DSM IP ASSETS B.V., Heerlen (NL)

(72) Inventors: Jacobus Thomas PRONK, Schipluiden (NL); Antonius Jeroen Adriaan VAN MARIS, Stockholm (SE); Victor Gabriel GUADALUPE MEDINA, Delft (NL)

(21) Appl. No.: 19/327,237

(22) Filed: Sep. 12, 2025

Related U.S. Application Data
(63) Continuation of application No. 17/531,218, filed on Nov. 19, 2021, now Pat. No. 12,435,339.
Application No. 17/531,218 is a continuation of application No. 16/833,342, filed on Mar. 27, 2020, now Pat. No. 11,214,810.
Application No. 16/833,342 is a continuation of application No. 15/970,641, filed on May 3, 2018, now Pat. No. 10,738,317.
Application No. 15/970,641 is a continuation of application No. 15/154,199, filed on May 13, 2016, now abandoned.
Application No. 15/154,199 is a continuation of application No. 14/189,989, filed on Feb. 25, 2014, now Pat. No. 9,528,117.
Application No. 14/189,989 is a continuation of application No. 13/061,695, filed on Jul. 18, 2011, now Pat. No. 8,795,998.

(30) Foreign Application Priority Data
Jul. 24, 2009 (EP) 09166360.9

(51) Int. Cl. C12N 15/81; C12N 1/18; C12N 9/00; C12N 9/02; C12N 9/04; C12P 7/06; C12P 7/10

(52) U.S. Cl. CPC C12N 15/815; C12N 1/18; C12N 9/0006; C12N 9/0008; C12N 9/93; C12P 7/06; C12P 7/10; C12Y 101/01001; C12Y 101/01008; C12Y 102/0101; C12Y 602/01001; Y02E 50/10; Y02P 20/52

(57) ABSTRACT

The present invention relates to a yeast cell, in particular a recombinant yeast cell, the cell lacking enzymatic activity needed for the NADH-dependent glycerol synthesis or the cell having a reduced enzymatic activity with respect to the NADH-dependent glycerol synthesis compared to its corresponding wild-type yeast cell, the cell comprising one or more heterologous nucleic acid sequences encoding an NAD+-dependent acetylating acetaldehyde dehydrogenase (EC 1.2.1.10) activity. The invention further relates to the use of a cell according to the invention in the preparation of ethanol.

### CROSS-REFERENCE TO RELATED APPLICATIONS

This application is a continuation of U.S. patent application Ser. No. 17/531,218, filed 19 Nov. 2021, which is a continuation of U.S. patent application Ser. No. 16/833,342, filed 27 Mar. 2020, issued as U.S. Pat. No. 11,214,810 on 4 Jan. 2022, which is a continuation of U.S. patent application Ser. No. 15/970,641, filed 3 May 2018, issued as U.S. Pat. No. 10,738,317 on 11 Aug. 2020, which is a continuation of abandoned U.S. patent application Ser. No. 15/154,199, filed 13 May 2016, which is a continuation of U.S. patent application Ser. No. 14/189,989, filed 25 Feb. 2014, issued as U.S. Pat. No. 9,528,117 on 27 Dec. 2016, which is a continuation of U.S. patent application Ser. No. 13/061,695, filed on 18 Jul. 2011, issued as U.S. Pat. No. 8,795,998 on 5 Aug. 2014, which is a National Stage entry of expired International Application No. PCT/NL2010/050475, filed 23 Jul. 2010, which claims priority to European Patent Application No. 09166360.9, filed 24 Jul. 2009, the entire contents of each of which are incorporated herein by reference.

### REFERENCE TO SEQUENCE LISTING XML

Pursuant to the EFS-Web legal framework and 37 C.F.R. § 1.831-835 (see M.P.E.P. § 2413.03), a Sequence Listing in extensible Markup Language (XML) format (entitled “Sequence_Listing_2919208-306010.xml” created on 12 Sep. 2025 and 53,023 bytes in size) is submitted concurrently with the instant application, and the entire contents of the Sequence Listing are incorporated herein by reference.

### BACKGROUND OF THE DISCLOSURE

The present invention relates to a recombinant yeast cell having the ability to produce a desired fermentation product, to the construction of said yeast cell by genetic modification and to a process for producing a fermentation product wherein said yeast cell is used.

Ethanol production by Saccharomyces cerevisiae is currently, by volume, the single largest fermentation process in industrial biotechnology. A global research effort is underway to expand the substrate range of S. cerevisiae to include lignocellulosic hydrolysates, in particular hydrolysed lignocellulosic biomass from non-food feedstocks (e.g. energy crops and agricultural residues, forestry residues or industrial/consumer waste materials that are rich in cellulose, hemicellulose and/or pectin) and to increase productivity, robustness and product yield.

Lignocellulosic biomass is abundant, however is in general not readily fermented by wild-type ethanol producing micro-organisms, such as S. cerevisiae. The biomass has to be hydrolysed. The resultant hydrolysate is often a mixture of various monosaccharides and oligosaccharides, which may not all be suitable substrates for the wild-type micro-organism. Further, the hydrolysates typically comprise acetic acid, formed as a by-product in particular when hydrolysing pectin or hemicellulose, and—dependent on the type of hydrolysis—one or more other by-products or residual reagents that may adversely affect the fermentation. In particular, acetic acid has been reported to negatively affect the kinetics and/or stoichiometry of sugar fermentation by wild-type and genetically modified S. cerevisiae strains and its toxicity is strongly augmented at low culture pH (Helle et al. Enzyme Microb Technol 33 (2003) 786-792; Bellissimi et al. FEMS Yeast Res 9 (2009) 358-364).

Various approaches have been proposed to improve the fermentative properties of ethanol producing organisms by genetic modification, and to improve the hydrolysis process of the biomass. E.g. an overview of developments in the fermentative production of ethanol from biomass hydrolysates is given in a review by A. van Maris et al. (Antonie van Leeuwenhoek (2006) 90:391-418). Reference is made to various ways in which S. cerevisiae may be modified and to various methods of hydrolysing lignocellulosic biomass.

A major challenge relating to the stoichiometry of yeast-based ethanol production is that substantial amounts of glycerol are invariably formed as a by-product. It has been estimated that, in typical industrial ethanol processes, up to about 4 wt. % of the sugar feedstock is converted into glycerol (Nissen et al. Yeast 16 (2000) 463-474). Under conditions that are ideal for anaerobic growth, the conversion into glycerol may even be higher, up to about 10%.

Glycerol production under anaerobic conditions is primarily linked to redox metabolism. During anaerobic growth of S. cerevisiae, sugar dissimilation occurs via alcoholic fermentation. In this process, the NADH formed in the glycolytic glyceraldehyde-3-phosphate dehydrogenase reaction is reoxidized by converting acetaldehyde, formed by decarboxylation of pyruvate to ethanol via NAD⁺-dependent alcohol dehydrogenase. The fixed stoichiometry of this redox-neutral dissimilatory pathway causes problems when a net reduction of NAD⁺ to NADH occurs elsewhere in metabolism. Under anaerobic conditions, NADH reoxidation in S. cerevisiae is strictly dependent on reduction of sugar to glycerol. Glycerol formation is initiated by reduction of the glycolytic intermediate dihydroxyacetone phosphate to glycerol 3-phosphate, a reaction catalyzed by NAD⁺-dependent glycerol 3-phosphate dehydrogenase. Subsequently, the glycerol 3-phosphate formed in this reaction is hydrolysed by glycerol-3-phosphatase to yield glycerol and inorganic phosphate. Consequently, glycerol is a major by-product during anaerobic production of ethanol by S. cerevisiae, which is undesired as it reduces overall conversion of sugar to ethanol. Further, the presence of glycerol in effluents of ethanol production plants may impose costs for waste-water treatment.

It is an object of the invention to provide a novel recombinant cell, which is suitable for the anaerobic, fermentative production of ethanol from a carbohydrate, in particular a carbohydrate obtained from lignocellulosic biomass, which has a reduced glycerol production compared to its corresponding wild-type organism or which lacks glycerol production if the cell is used for the fermentative preparation of ethanol.

It is further an object to provide a novel method for fermentatively preparing ethanol in anaerobic yeast cultures, in which method no glycerol is formed, or at least wherein less glycerol is formed than in a method making use of known strains of S. cerevisiae.

One or more further objects that may be met are apparent from the description and/or claims.

The inventors have realised that it is possible to meet one or more of these objectives by providing a specific recombinant cell wherein a specific other enzymatic activity has been incorporated, which allows re-oxidation of NADH formed in the fermentation of a carbohydrate, also in the absence of enzymatic activity needed for the NADH-dependent glycerol synthesis.

Accordingly the present invention relates to a recombinant yeast cell, the cell comprising one or more recombinant, in particular one or more heterologous, nucleic acid sequences encoding an NAD⁺-dependent acetylating acetaldehyde dehydrogenase (EC 1.2.1.10) activity.

The inventors have in particular realised that it is advantageous to provide a cell without enzymatic activity needed for the NADH-dependent glycerol synthesis or a cell with reduced enzymatic activity needed for the NADH-dependent glycerol synthesis.

Accordingly, the invention in particular relates to a recombinant yeast cell comprising one or more heterologous nucleic acid sequences encoding an NAD⁺-dependent acetylating acetaldehyde dehydrogenase activity, wherein the cell lacks enzymatic activity needed for the NADH-dependent glycerol synthesis (i.e. is free of such activity), or wherein the cell has a reduced enzymatic activity with respect to NADH-dependent glycerol synthesis compared to its corresponding wild-type yeast cell.

The invention is further directed to the use of a cell according to the invention for the preparation of ethanol.

In particular, the invention is further directed to a method for preparing ethanol, comprising preparing ethanol from a fermentable carbohydrate and from acetate, which preparation is carried out under anaerobic fermentative conditions using a yeast cell, said cell expressing acetyl-Coenzyme A synthetase activity and NAD⁺-dependent acetylating acetaldehyde dehydrogenase activity, said cell preferably lacking enzymatic activity needed for the biochemical pathway for glycerol synthesis from a carbohydrate or having a reduced enzymatic activity with respect to the biochemical pathway for glycerol synthesis from a carbohydrate compared to a wild-type S. cerevisiae cell.

Advantageously, in accordance with the invention ethanol is produced in a molar ratio of glycerol: ethanol of less than 0.04:1, in particular of less than 0.02:1, preferably of less than 0.01:1. Glycerol production may be absent (undetectable), although at least in some embodiments (wherein NADH-dependent glycerol synthesis is reduced yet not completely prohibited) some glycerol may be produced as a side-product, e.g. in a ratio glycerol to ethanol of 0.001:1 or more.

### BRIEF DESCRIPTION OF THE DRAWINGS

FIG. 1 schematically shows a genetic modification procedure which may be carried out as part of the production of a cell according to the invention.

FIGS. 2A-2D show concentrations of biomass and products in anaerobic batch cultures of different S. cerevisiae strains on glucose (20 g l⁻¹). Acetic acid (2.0 g l⁻¹) was present from the start of the fermentation (FIGS. 2A & 2B) or added at the time point indicated by the arrow (FIGS. 2C & 2D). Growth conditions: T=30 ° C., pH 5.0. Symbols: ▴, optical density at 660 nm; ●, glucose; ○, ethanol; ▪, acetate; □, glycerol. Each graph represents values from one of two independent replicates, yielding data that differed by less than 5%. FIG. 2A: S. cerevisiae IME076 (GPD1 GPD2). FIG. 2B: S. cerevisiae IMZ132 (gpd1Δ gpd2Δ overexpressing the E. coli mhpF gene). FIG. 2C: S. cerevisiae IMZ132 (gpd1Δ gpd2Δ overexpressing the E. coli mhpF gene). FIG. 2D: S. cerevisiae IMZ127 (gpd1Δ gpd2Δ) grown on glucose (20 g·l⁻¹).

FIG. 3 shows the volumetric CO2 percentage present in the out flow of a batch fermentation inoculated with strain IMZ130 (gpd1Δ gpd2ΔmpF). The graph is presented in a logarithmic scale on the y-axis in order to demonstrate exponential growth and calculate the maximum specific growth rate.

### DETAILED DESCRIPTION OF THE DISCLOSURE

The present invention allows complete elimination of glycerol production, or at least a significant reduction thereof, by providing a recombinant yeast cell, in particular S. cerevisiae, such that it can reoxidise NADH by the reduction of acetic acid to ethanol via NADH-dependent reactions.

This is not only advantageous in that glycerol production is avoided or at least reduced, but since the product formed in the re-oxidation of NADH is also the desired product, namely ethanol, a method of the invention may also offer an increased product yield (determined as the wt. % of converted feedstock, i.e. carbohydrate plus acetic acid, that is converted into ethanol).

Since acetic acid is generally available at significant amounts in lignocellulosic hydrolysates, this makes the present invention particularly advantageous for the preparation of ethanol using lignocellulosic biomass as a source for the fermentable carbohydrate. Further, carbohydrate sources that may contain a considerable amount of acetate include sugar beet molasses (hydrolysates of) and starch containing (e.g. waste products from corn dry milling processes, from corn wet milling processes; from starch wastes processes, e.g. with stillage recycles). The invention contributes to a decrease of the levels of the inhibiting compound acetic acid and a larger fraction of the hydrolysate actually becomes a substrate for the production of the ethanol.

Good results have been achieved with a yeast cell without noticeable enzymatic activity needed for the NADH-dependent glycerol synthesis, as illustrated in the example. However, the inventors contemplate that also a yeast cell according to the invention having NADH-dependent glycerol synthesis activity may advantageously be used for, e.g., ethanol production. It is contemplated that such cell can use acetate to re-oxidise at least part of the NADH. Thereby the acetate may compete with the NADH-dependent glycerol synthesis pathway and thus potentially reduce the glycerol synthesis. Morover, acetate present in a feedstock used for the production of ethanol, such as a lignocellulosic hydrolysate, can be converted into ethanol, thereby increasing product yield.

The term “a” or “an” as used herein is defined as “at least one” unless specified otherwise.

When referring to a noun (e.g. a compound, an additive, etc.) in the singular, the plural is meant to be included. Thus, when referring to a specific moiety, e.g. “compound”, this means “at least one” of that moiety, e.g. “at least one compound”, unless specified otherwise.

The term ‘or’ as used herein is to be understood as ‘and/or’.

When referred herein to a carboxylate, e.g. acetate, the corresponding carboxylic acid (its conjugated acid) as well as a salt thereof is meant to be included, and vice versa.

When referring to a compound of which several isomers exist (e.g. a D and an L enantiomer), the compound in principle includes all enantiomers, diastereomers and cis/trans isomers of that compound that may be used in the particular method of the invention; in particular when referring to such as compound, it includes the natural isomer(s).

The term ‘fermentation’, ‘fermentative’ and the like is used herein in a classical sense, i.e. to indicate that a process is or has been carried out under anaerobic conditions. Anaerobic conditions are herein defined as conditions without any oxygen or in which essentially no oxygen is consumed by the yeast cell, in particular a yeast cell, and usually corresponds to an oxygen consumption of less than 5 mmol/l·h, in particular to an oxygen consumption of less than 2.5 mmol/l·h, or less than 1 mmol/l·h. This usually corresponds to a dissolved oxygen concentration in the culture broth of less than 5% of air saturation, in particular to a dissolved oxygen concentration of less than 1% of air saturation, or less than 0.2% of air saturation.

The term “yeast” or “yeast cell” refers to a phylogenetically diverse group of single-celled fungi, most of which are in the division of Ascomycota and Basidiomycota. The budding yeasts (“true yeasts”) are classified in the order Saccharomycesles, with Saccharomyces cerevisiae as the most well-known species.

The term “recombinant (cell)” as used herein, refers to a strain (cell) containing nucleic acid which is the result of one or more genetic modifications using recombinant DNA technique(s) and/or another mutagenic technique(s). In particular a recombinant cell may comprise nucleic acid not present in a corresponding wild-type cell, which nucleic acid has been introduced into that strain (cell) using recombinant DNA techniques (a transgenic cell), or which nucleic acid not present in said wild-type is the result of one or more mutations—for example using recombinant DNA techniques or another mutagenesis technique such as UV-irradiation—in a nucleic acid sequence present in said wild-type (such as a gene encoding a wild-type polypeptide) or wherein the nucleic acid sequence of a gene has been modified to target the polypeptide product (encoding it) towards another cellular compartment. Further, the term “recombinant (cell)” in particular relates to a strain (cell) from which DNA sequences have been removed using recombinant DNA techniques.

The term “transgenic (yeast) cell” as used herein, refers to a strain (cell) containing nucleic acid not naturally occurring in that strain (cell) and which has been introduced into that strain (cell) using recombinant DNA techniques, i.e. a recombinant cell).

The term “mutated” as used herein regarding proteins or polypeptides means that at least one amino acid in the wild-type or naturally occurring protein or polypeptide sequence has been replaced with a different amino acid, inserted or deleted from the sequence via mutagenesis of nucleic acids encoding these amino acids. Mutagenesis is a well-known method in the art, and includes, for example, site-directed mutagenesis by means of PCR or via oligonucleotide-mediated mutagenesis as described in Sambrook et al., Molecular Cloning-A Laboratory Manual, 2nd ed., Vol. 1-3 (1989). The term “mutated” as used herein regarding genes means that at least one nucleotide in the nucleic acid sequence of that gene or a regulatory sequence thereof, has been replaced with a different nucleotide, or has been deleted from the sequence via mutagenesis, resulting in the transcription of a protein sequence with a qualitatively of quantitatively altered function or the knock-out of that gene.

The term “gene”, as used herein, refers to a nucleic acid sequence containing a template for a nucleic acid polymerase, in eukaryotes, RNA polymerase II. Genes are transcribed into mRNAs that are then translated into protein.

The term “nucleic acid” as used herein, includes reference to a deoxyribonucleotide or ribonucleotide polymer, i.e. a polynucleotide, in either single or double-stranded form, and unless otherwise limited, encompasses known analogues having the essential nature of natural nucleotides in that they hybridize to single-stranded nucleic acids in a manner similar to naturally occurring nucleotides (e. g., peptide nucleic acids). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulphation, gamma-carboxylation of glutamic acid residues, hydroxylation and ADP-ribosylation.

When an enzyme is mentioned with reference to an enzyme class (EC), the enzyme class is a class wherein the enzyme is classified or may be classified, on the basis of the Enzyme Nomenclature provided by the Nomenclature Committee of the International Union of Biochemistry and Molecular Biology (NC-IUBMB), which nomenclature may be found at chem.qmul.ac.uk/iubmb/enzyme. Other suitable enzymes that have not (yet) been classified in a specified class but may be classified as such, are meant to be included.

If referred herein to a protein or a nucleic acid sequence, such as a gene, by reference to a accession number, this number in particular is used to refer to a protein or nucleic acid sequence (gene) having a sequence as can be found via ncbi.nlm.nih.gov, (as available on 13 Jul. 2009) unless specified otherwise.

Every nucleic acid sequence herein that encodes a polypeptide also, by reference to the genetic code, describes every possible silent variation of the nucleic acid. The term “conservatively modified variants” applies to both amino acid and nucleic acid sequences. With respect to particular nucleic acid sequences, conservatively modified variants refers to those nucleic acids which encode identical or conservatively modified variants of the amino acid sequences due to the degeneracy of the genetic code. The term “degeneracy of the genetic code” refers to the fact that a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation.

The term “functional homologue” (or in short “homologue”) of a polypeptide having a specific sequence (e.g. SEQ ID NO:2), as used herein, refers to a polypeptide comprising said specific sequence with the proviso that one or more amino acids are substituted, deleted, added, and/or inserted, and which polypeptide has (qualitatively) the same enzymatic functionality for substrate conversion, for instance an NAD⁺-dependent acetylating acetaldehyde dehydrogenase (EC 1.2.1.10) activity homologue is capable of converting acetaldehyde into ethanol. This functionality may be tested by use of an assay system comprising a recombinant yeast cell comprising an expression vector for the expression of the homologue in yeast, said expression vector comprising a heterologous nucleic acid sequence operably linked to a promoter functional in the yeast and said heterologous nucleic acid sequence encoding the homologous polypeptide of which enzymatic activity for converting acetyl-Coenzyme A to acetaldehyde in the yeast cell is to be tested, and assessing whether said conversion occurs in said cells. Candidate homologues may be identified by using in silico similarity analyses. A detailed example of such an analysis is described in Example 2 of WO2009/013159. The skilled person will be able to derive there from how suitable candidate homologues may be found and, optionally upon codon (pair) optimization, will be able to test the required functionality of such candidate homologues using a suitable assay system as described above. A suitable homologue represents a polypeptide having an amino acid sequence similar to a specific polypeptide of more than 50%, preferably of 60% or more, in particular of at least 70%, more in particular of at least 80%, at least 90%, at least 95%, at least 97%, at least 98% or at least 99%, for instance having such an amino acid sequence similarity to SEQ ID NO:2, and having the required enzymatic functionality for converting acetyl-Coenzyme A to acetaldehyde. With respect to nucleic acid sequences, the term functional homologue is meant to include nucleic acid sequences which differ from another nucleic acid sequence due to the degeneracy of the genetic code and encode the same polypeptide sequence.

Sequence identity is herein defined as a relationship between two or more amino acid (polypeptide or protein) sequences or two or more nucleic acid (polynucleotide) sequences, as determined by comparing the sequences. Usually, sequence identities or similarities are compared over the whole length of the sequences compared. In the art, “identity” also means the degree of sequence relatedness between amino acid or nucleic acid sequences, as the case may be, as determined by the match between strings of such sequences.

Preferred methods to determine identity are designed to give the largest match between the sequences tested. Methods to determine identity and similarity are codified in publicly available computer programs. Preferred computer program methods to determine identity and similarity between two sequences include e.g. the BestFit, BLASTP, BLASTN, and FASTA (Altschul, S. F. et al., J. Mol. Biol. 215:403-410 (1990), publicly available from NCBI and other sources (BLAST Manual, Altschul, S., et al., NCBI NLM NIH Bethesda, MD 20894). Preferred parameters for amino acid sequences comparison using BLASTP are gap open 11.0, gap extend 1, Blosum 62 matrix.

“Expression” refers to the transcription of a gene into structural RNA (rRNA, tRNA) or messenger RNA (mRNA) with subsequent translation into a protein.

As used herein, “heterologous” in reference to a nucleic acid or protein is a nucleic acid or protein that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

The term “heterologous expression” refers to the expression of heterologous nucleic acids in a host cell. The expression of heterologous proteins in eukaryotic host cell systems such as yeast are well known to those of skill in the art. A polynucleotide comprising a nucleic acid sequence of a gene encoding an enzyme with a specific activity can be expressed in such a eukaryotic system. In some embodiments, transformed/transfected yeast cells may be employed as expression systems for the expression of the enzymes. Expression of heterologous proteins in yeast is well known. Sherman, F., et al., Methods in Yeast Genetics, Cold Spring Harbor Laboratory (1982) is a well recognized work describing the various methods available to express proteins in yeast. Two widely utilized yeasts are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

As used herein “promoter” is a DNA sequence that directs the transcription of a (structural) gene. Typically, a promoter is located in the 5′ region of a gene, proximal to the transcriptional start site of a (structural) gene. Promoter sequences may be constitutive, inducible or repressible. If a promoter is an inducible promoter, then the rate of transcription increases in response to an inducing agent.

The term “vector” as used herein, includes reference to an autosomal expression vector and to an integration vector used for integration into the chromosome.

The term “expression vector” refers to a DNA molecule, linear or circular, that comprises a segment encoding a polypeptide of interest under the control of (i.e. operably linked to) additional nucleic acid segments that provide for its transcription. Such additional segments may include promoter and terminator sequences, and may optionally include one or more origins of replication, one or more selectable markers, an enhancer, a polyadenylation signal, and the like. Expression vectors are generally derived from plasmid or viral DNA, or may contain elements of both. In particular an expression vector comprises a nucleic acid sequence that comprises in the 5′ to 3′ direction and operably linked: (a) a yeast-recognized transcription and translation initiation region, (b) a coding sequence for a polypeptide of interest, and (c) a yeast-recognized transcription and translation termination region. “Plasmid” refers to autonomously replicating extrachromosomal DNA which is not integrated into a microorganism's genome and is usually circular in nature.

An “integration vector” refers to a DNA molecule, linear or circular, that can be incorporated in a microorganism's genome and provides for stable inheritance of a gene encoding a polypeptide of interest. The integration vector generally comprises one or more segments comprising a gene sequence encoding a polypeptide of interest under the control of (i.e. operably linked to) additional nucleic acid segments that provide for its transcription. Such additional segments may include promoter and terminator sequences, and one or more segments that drive the incorporation of the gene of interest into the genome of the target cell, usually by the process of homologous recombination. Typically, the integration vector will be one which can be transferred into the target cell, but which has a replicon which is nonfunctional in that organism. Integration of the segment comprising the gene of interest may be selected if an appropriate marker is included within that segment.

As used herein, the term “operably linked” refers to a juxtaposition wherein the components so described are in a relationship permitting them to function in their intended manner. A control sequence “operably linked” to another control sequence and/or to a coding sequence is ligated in such a way that transcription and/or expression of the coding sequence is achieved under conditions compatible with the control sequence. Generally, operably linked means that the nucleic acid sequences being linked are contiguous and, where necessary to join two protein coding regions, contiguous and in the same reading frame.

By “host cell” is meant a cell which contains a vector and supports the replication and/or expression of the vector. Host cells may be prokaryotic cells such as E. coli, or eukaryotic cells such as yeast, insect, amphibian, or mammalian cells. Preferably, host cells are cells of the order of Actinomycetales, most preferably yeast cells, most preferably cells of Saccharomyces cerevisiae.

“Transformation” and “transforming”, as used herein, refers to the insertion of an exogenous polynucleotide into a host cell, irrespective of the method used for the insertion, for example, direct uptake, transduction, f-mating or electroporation. The exogenous polynucleotide may be maintained as a non-integrated vector, for example, a plasmid, or alternatively, may be integrated into the host cell genome.

A cell according to the invention preferably is selected from the group of Saccharomycesceae, more preferably from the group of Saccharomyces cells, Zygosaccharomyces and Kluyveromyces cells. In particular good results have been achieved with a Saccharomyces cerevisiae host cell. Zygosaccharomyces baillii is another particularly preferred cell, especially for its high acetate tolerance and its high ethanol tolerance.

Further, a cell according to the invention may be a yeast cell selected from the group of xylose-fermenting yeasts, more preferably a Pichia species, for example Pichia stipitis or Pichia angusta (also known as Hansenula polymorpha).

In a further embodiment, a host cell according to the invention is a host cell that naturally lacks enzymatic activity needed for the NADH-dependent glycerol synthesis, for example yeast cells belonging to the species Brettanomyces intermedius.

A preferred cell according to the invention is free of enzymatic activity needed for the NADH-dependent glycerol synthesis or has a reduced enzymatic activity with respect to the NADH-dependent biochemical pathway for glycerol synthesis from a carbohydrate compared to its corresponding wild-type yeast cell.

A reduced enzymatic activity can be achieved by modifying one or more genes encoding a NAD-dependent glycerol 3-phosphate dehydrogenase activity (GPD) or one or more genes encoding a glycerol phosphate phosphatase activity (GPP), such that the enzyme is expressed considerably less than in the wild-type or such that the gene encoded a polypeptide with reduced activity. Such modifications can be carried out using commonly known biotechnological techniques, and may in particular include one or more knock-out mutations or site-directed mutagenesis of promoter regions or coding regions of the structural genes encoding GPD and/or GPP. Alternatively, yeast strains that are defective in glycerol production may be obtained by random mutagenesis followed by selection of strains with reduced or absent activity of GPD and/or GPP. S. cerevisiae GDP 1, GDP2, GPP1 and GPP2 genes are shown in SEQ ID NO:24-27.

Preferably at least one gene encoding a GPD or at least one gene encoding a GPP is entirely deleted, or at least a part of the gene is deleted that encodes a part of the enzyme that is essential for its activity. In particular, good results have been achieved with a S. cerevisiae cell, wherein the open reading frames of the GPD1 gene and of the GPD2 gene have been inactivated. Inactivation of a structural gene (target gene) can be accomplished by a person skilled in the art by synthetically synthesizing or otherwise constructing a DNA fragment consisting of a selectable marker gene flanked by DNA sequences that are identical to sequences that flank the region of the host cell's genome that is to be deleted. In particular, good results have been obtained with the inactivation of the GPD1 and GPD2 genes in Saccharomyces cerevisiae by integration of the marker genes kanMX and hphMX4. Subsequently this DNA fragment is transformed into a host cell. Transformed cells that express the dominant marker gene are checked for correct replacement of the region that was designed to be deleted, for example by a diagnostic polymerase chain reaction or Southern hybridization.

As indicated above, a cell according to the invention comprises a heterologous nucleic acid sequence encoding an NAD⁺-dependent, acetylating acetaldehyde dehydrogenase (EC 1.2.1.10). This enzyme catalyses the conversion of acetyl-Coenzyme A to acetaldehyde. This conversion can be represented by the equilibrium reaction formula:
acetyl-Coenzyme A⁺NADH⁺H⁺<->acetaldehyde⁺NAD⁺⁺Coenzyme A.

Thus, this enzyme allows the re-oxidation of NADH when acetyl-Coenzyme A is generated from acetate present in the growth medium, and thereby glycerol synthesis is no longer needed for redox cofactor balancing.

The nucleic acid sequence encoding the NAD⁺-dependent acetylating acetaldehyde dehydrogenase may in principle originate from any organism comprising a nucleic acid sequence encoding said dehydrogenase.

Known NAD⁺-dependent acetylating acetaldehyde dehydrogenases that can catalyse the NADH-dependent reduction of acetyl-Coenzyme A to acetaldehyde may in general be divided in three types of NAD⁺-dependent acetylating acetaldehyde dehydrogenase functional homologues:

1) Bifunctional proteins that catalyse the reversible conversion of acetyl-Coenzyme A to acetaldehyde, and the subsequent reversible conversion of acetaldehyde to ethanol. An example of this type of proteins is the AdhE protein in E. coli (Gen Bank No: NP_415757). AdhE appears to be the evolutionary product of a gene fusion. The NH2-terminal region of the AdhE protein is highly homologous to aldehyde:NAD⁺ oxidoreductases, whereas the COOH-terminal region is homologous to a family of Fe2⁺-dependent ethanol:NAD⁺ oxidoreductases (Membrillo-Hernandez et al., (2000) J. Biol. Chem. 275:33869-33875). The E. coli AdhE is subject to metal-catalyzed oxidation and therefore oxygen-sensitive (Tamarit et al. (1998) J. Biol. Chem. 273:3027-32).

2) Proteins that catalyse the reversible conversion of acetyl-Coenzyme A to acetaldehyde in strictly or facultative anaerobic micro-organisms but do not possess alcohol dehydrogenase activity. An example of this type of proteins has been reported in Clostridium kluyveri (Smith et al. (1980) Arch. Biochem. Biophys. 203:663-675). An acetylating acetaldehyde dehydrogenase has been annotated in the genome of Clostridium kluyveri DSM 555 (GenBank No: EDK33116). A homologous protein AcdH is identified in the genome of Lactobacillus plantarum (GenBank No: NP_784141). Another example of this type of proteins is the said gene product in Clostridium beijerinckii NRRL B593 (Toth et al. (1999) Appl. Environ. Microbiol. 65:4973-4980, GenBank No: AAD31841).

3) Proteins that are part of a bifunctional aldolase-dehydrogenase complex involved in 4-hydroxy-2-ketovalerate catabolismuch bifunctional enzymes catalyze the final two steps of the meta-cleavage pathway for catechol, an intermediate in many bacterial species in the degradation of phenols, toluates, naphthalene, biphenyls and other aromatic compounds (Powlowski and Shingler (1994) Biodegradation 5, 219-236). 4-Hydroxy-2-ketovalerate is first converted by 4-hydroxy-2-ketovalerate aldolase to pyruvate and acetaldehyde, subsequently acetaldehyde is converted by acetylating acetaldehyde dehydrogenase to acetyl-CoA. An example of this type of acetylating acetaldehyde dehydrogenase is the DmpF protein in Pseudomonas sp CF600 (GenBank No: CAA43226) (Shingler et al. (1992) J. Bacteriol. 174:71 1-24). The E. coli MphF protein (Ferrandez et al. (1997) J. Bacteriol. 179:2573-2581, GenBank No: NP_414885) is homologous to the DmpF protein in Pseudomonas sp. CF600.

A suitable nucleic acid sequence may in particular be found in an organism selected from the group of Escherichia, in particular E. coli; Mycobacterium, in particular Mycobacterium marinum, Mycobacterium ulcerans, Mycobacterium tuberculosis; Carboxydothermus, in particular Carboxydothermus hydrogenoformans; Entamoeba, in particular Entamoeba histolytica; Shigella, in particular Shigella sonnei; Burkholderia, in particular Burkholderia pseudomallei, Klebsiella, in particular Klebsiella pneumoniae; Azotobacter, in particular Azotobacter vinelandii; Azoarcus sp; Cupriavidus, in particular Cupriavidus taiwanensis; Pseudomonas, in particular Pseudomonas sp. CF600; Pelomaculum, in particular Pelotomaculum thermopropionicum. Preferably, the nucleic acid sequence encoding the NAD⁺-dependent acetylating acetaldehyde dehydrogenase originates from Escherichia, more preferably from E. coli.

Particularly suitable is an mhpF gene from E. coli, or a functional homologue thereof. This gene is described in Ferrandez et al. (1997) J. Bacteriol. 179:2573-2581. Good results have been obtained with S. cerevisiae, wherein an mhpF gene from E. coli has been incorporated.

In a further advantageous embodiment the nucleic acid sequence encoding an (acetylating) acetaldehyde dehydrogenase is from, in particular Pseudomonas. dmpF from Pseudomonas sp. CF600.

In principle, the nucleic acid sequence encoding the NAD⁺-dependent, acetylating acetaldehyde dehydrogenase may be a wild type nucleic acid sequence.

A preferred nucleic acid sequence encodes the NAD⁺-dependent, acetylating acetaldehyde dehydrogenase represented by SEQ ID NO:2, SEQ ID NO:29, or a functional homologue of SEQ ID NO:2 or SEQ ID NO:29. In particular the nucleic acid sequence comprises a sequence according to SEQ ID NO:1. SEQ ID NO:28 or a functional homologue of SEQ ID NO:1 or SEQ ID NO:28.

Further, an acetylating acetaldehyde dehydrogenase (or nucleic acid sequence encoding such activity) may in for instance be selected from the group of Escherichia coli adhE, Entamoeba histolytica adh2, Staphylococcus aureus adhE, Piromyces sp. E2 adhE, Clostridium kluyveri EDK33116, Lactobacillus plantarum acdH, and Pseudomonas putida YP 001268189. For sequences of these enzymes, nucleic acid sequences encoding these enzymes and methodology to incorporate the nucleic acid sequence into a host cell, reference is made to WO 2009/013159, in particular Example 3, Table 1 (page 26) and the Sequence ID numbers mentioned therein, of which publication Table 1 and the sequences represented by the Sequence ID numbers mentioned in said Table are incorporated herein by reference.

Usually, a cell according to the invention also comprises an acetyl-Coenzyme A synthetase, which enzyme catalyses the formation of acetyl-coenzyme A from acetate. This enzyme may be present in the wild-type cell, as is for instance the case with S. cerevisiae which contains two acetyl-Coenzyme A synthetase isoenzymes encoded by the ACS1 [SEQ ID NO:17] and ACS2 [SEQ ID NO:18] genes (van den Berg et al (1996) J. Biol. Chem. 271:28953-28959), or a host cell may be provided with one or more heterologous gene(s) encoding this activity, e.g. the ACS1 and/or ACS2 gene of S. cerevisiae or a functional homologue thereof may be incorporated into a cell lacking acetyl-Coenzyme A synthetase isoenzyme activity.

Further, in particular in view of an efficient ethanol production, but also for an efficient NADH oxidation, it is preferred that the cell comprises an NAD⁺-dependent alcohol dehydrogenase (EC 1.1.1.1). This enzyme catalyses the conversion of acetaldehyde into ethanol. The cell may naturally comprise a gene encoding such a dehydrogenase, as is de case with S. cerevisiae (ADH1-5) [SEQ ID NO:19-23], see ‘Lutstorf and Megnet. 1968 Arch. Biochem. Biophys. 126:933-944’, or ‘Ciriacy, 1975, Mutat. Res. 29:315-326’), or a host cell may be provided with one or more heterologous gene(s) encoding this activity, e.g. any or each of the ADH1-5 genes of S. cerevisiae or functional homologues thereof may be incorporated into a cell lacking NAD⁺-dependent alcohol dehydrogenase activity.

Specifically preferred cells according to the invention are cells of the S. cerevisiae strain deposited on 16 Jul. 2009 at the Centraalbureau voor Schimmelcultures (Utrecht, the Netherlands) having deposit number CBS125049.

In specific aspect, the present invention is directed to a method of preparing a recombinant yeast cell according to the invention.

The genetic modification of a cell, comprising the incorporation of one or more heterologous nucleic acid sequences into a host cell, and usually comprising mutation (including complete deletion) of a gene encoding an enzymatic activity needed for the NADH-dependent glycerol synthesis, can be based on common general knowledge, e.g. by standard genetic and molecular biology techniques as generally known in the art and have been previously described (e.g. Maniatis et al. 1982 “Molecular cloning: a laboratory manual”. Cold Spring Harbor Laboratory, Cold Spring Harbor, N.Y.; Miller 1972 “Experiments in molecular genetics”, Cold Spring Harbor Laboratory, Cold Spring Harbor; Sambrook and Russell 2001 “Molecular cloning: a laboratory manual” (3rd edition), Cold Spring Harbor Laboratory, Cold Spring Harbor Laboratory Press; F. Ausubel et al., eds., “Current protocols in molecular biology”, Green Publishing and Wiley Interscience, New York 1987).

A method of the invention for preparing a recombinant yeast cell according to the invention, comprising
(a) providing a yeast cell, preferably a yeast cell selected from the group of yeast cells lacking enzymatic activity needed for the NADH-dependent glycerol synthesis and yeast cell having a reduced enzymatic activity with respect to glycerol synthesis compared to its corresponding wild-type yeast cell;
(b) obtaining a nucleic acid segment comprising a gene that is heterologous to said yeast cell and encodes an enzyme that has NAD⁺-dependent acetylating acetaldehyde dehydrogenase activity, and wherein said gene is operably linked to a promoter functional in said yeast cell;
(c) if desired (e.g. if the yeast cell lacks acetyl-Coenzyme A synthetase activity or in which the activity of acetyl-Coenzyme A synthetase(s) is limiting the overall in vivo activity of the pathway for conversion of acetate into ethanol or expresses acetyl-Coenzyme A synthetase in a cellular compartment that is not compatible with its use in the invention), obtaining a nucleic acid segment comprising a gene that is heterologous to said yeast cell and encodes an enzyme that has an acetyl-Coenzyme A synthetase activity, and wherein said gene is operably linked to a promoter functional in said yeast cell;
(d) if desired (e.g. if the yeast cell lacks NAD⁺-dependent alcohol dehydrogenase activity or in which NAD⁺-dependent alcohol dehydrogenase activity is limiting the in vivo activity of the pathway for conversion of acetate into ethanol or expresses NAD⁺-dependent alcohol dehydrogenase in a cellular compartment that is not compatible with its use in the invention), obtaining a nucleic acid segment comprising a gene that is heterologous to said yeast cell and encodes an enzyme that has an NAD⁺-dependent alcohol dehydrogenase activity, and wherein said gene is operably linked to a promoter functional in said yeast cell; and
(e) transforming a yeast cell with said nucleic acid segment or segments thereby providing a recombinant yeast cell which expresses said heterologous gene and wherein said recombinant yeast cell exhibits reduced NADH-dependent glycerol synthesis under fermentative conditions as compared to a corresponding non-recombinant yeast cell or wherein in said yeast cell NADH-dependent glycerol synthesis under fermentative conditions is absent.

Promoters for yeast cells are known in the art and can be, for example, the triosephosphate dehydrogenase TPI1 promoters, glyceraldehyde-3-phosphate dehydrogenase TDH3 promoters, translational elongation factor EF-1 alpha TEF1 promoters, the alcohol dehydrogenase ADH1 promoters, glucose-6-phosphate dehydrogenase gpdA and ZWF1 promoters, protease promoters such as pepA, pepB, pepC, the glucoamylase glaA promoters, amylase amyA, amyB promoters, the catalase catR or catA promoters, glucose oxidase goxC promoter, beta-galactosidase lacA promoter, alpha-glucosidase aglA promoter, translation elongation factor tefA promoter, xylanase promoters such as xlnA, xlnB, xlnC, xlnD, cellulase promoters such as eglA, eglB, cbhA, promoters of transcriptional regulators such as areA, creA, xlnR, pacC, prtT, etc or any other, and can be found among others at the NCBI website (ncbi.nlm.nih.gov/entrez).

In a preferred embodiment, the heterologous nucleic acid sequence to be introduced into a yeast cell when preparing a recombinant yeast cell of the invention, is incorporated into a vector, and the transformation of the cell is carried out with the vector. If more than one heterologous nucleic acid sequence (encoding different enzymatic activities or together encoding a single enzymatic activity) are to be incorporated, these nucleic acid sequences may be present in a single vector or these nucleic acid sequences may be incorporated as part of separate vectors.

Accordingly, the invention is further directed to a vector for the expression of a heterologous polypeptide in a yeast cell, in particular a yeast cell, said expression vector comprising one or more heterologous nucleic acid sequences operably linked to a promoter functional in the yeast cell and said heterologous nucleic acid sequence(s) encoding a polypeptide having enzymatic activity for converting acetyl-Coenzyme A into acetaldehyde in (the cytosol of) said yeast cell, wherein said polypeptide preferably comprises a sequence according to SEQ ID NO:2 or a functional homologue thereof.

The vector (used in a method of) the invention may be a phage vector, a bacterial vector, a centromeric, episomal or integrating plasmid vector or a viral vector.

In a preferred embodiment the vector is a vector for expression in Saccharomyces, in particular S. cerevisiae. In such embodiment, the heterologous nucleic acid sequence encoding NAD⁺-dependent acetylating acetaldehyde dehydrogenase activity may be codon(-pair) optimised for expression in Saccharomyces, in particular S. cerevisiae, although good results have been achieved with a wild-type encoding nucleic acid sequence.

In order to achieve optimal expression in a specific cell (such as S. cerevisiae), the codon (pair) usage of the heterologous gene may be optimized by using any one of a variety of synthetic gene design software packages, for instance GeneOptimizer® from Geneart AG (Regensburg, Germany) for codon usage optimization or codon pair usage optimization as described in PCT/EP2007/05594. Such adaptation of codon usage ensures that the heterologous genes, which are for instance of bacterial origin, are effectively processed by the yeast transcription and translation machinery. Optimisation of codon pair usage will result in enhanced protein expression in the yeast cell. The optimised sequences may for instance be cloned into a high copy yeast expression plasmid, operably linked to a (preferably constitutive) promoter functional in a fungus (yeast).

As indicated above, the invention is further directed to the preparation of ethanol.

For a method of preparing ethanol, a cell according to the invention is used which under anaerobic conditions ferments a sugar thereby forming ethanol Suitable yeast cells for fermenting the sugar are generally known and include amongst others S. cerevisiae. In a method of the invention, a yeast cell is used that also produces an NAD⁺-dependent acetylating acetaldehyde dehydrogenase (EC 1.2.1.10). This cell can be obtained as described above, and illustrated in the example herein below. If used for the preparation of ethanol, the cell preferably also includes an acetyl-Coenzyme A synthetase activity. If used for the preparation of ethanol, the cell preferably also includes an NAD⁺-dependent alcohol dehydrogenase activity. These activities may be naturally present, as in S. cerevisiae, or provided by genetic modification (see also herein above).

The fermentation conditions can in principle be based on generally known conditions, e.g. as described in the review by Van Maris cited above, or the references cited therein, with the proviso that typically the medium wherein the fermentation is carried comprises acetate in addition to the fermentable carbohydrate(s).

The molar ratio acetate to carbohydrate consumed by anaerobic cultures of the yeast cells modified according to the invention is usually at least 0.004, in particular at least 0.01, at least 0.03, at least 0.1, or at least 0.2. The molar ratio acetate to carbohydrate present in hydrolysates of lignocellulosic biomass is usually less than 0.70, in particular 0.5 or less, more in particular 0.3 or less, or 0.2 or less. Herein the number of moles of carbohydrate is based on monosaccharide units, i.e. one mole of a oligo/polysaccharide having n monosaccharide units counts as n mol.

In absolute terms, the fermentable carbohydrate concentration is usually in the range of 65 to 400 g/L, in particular in the range of 100 to 250 g/L.

In absolute terms, the acetate concentration is usually in the range of 0.5 to 20 g/L, in particular in the range of 1-15 g/L, more in particular in the range of 2 to 12 g/L.

The pH can be chosen dependent on what is acceptable to the organism that is used, based on common general knowledge or can be routinely determined. Usually the fermentation is carried out at a neutral or acidic pH, in particular at a pH in the range of 2-7, more in particular at a pH of 3-6, even more in particular 3.5-5.5 (apparent pH as measured in the fermentation medium at the temperature at which fermentation takes place).

The temperature can be chosen dependent on what is acceptable to the organism that is used. Usually the temperature is in the range of 15-50 degrees C., in particular in the range of 25-45 degrees C.

As a fermentable carbohydrate in principle any carbohydrate can be used that is metabolised by the specific recombinant cell, with ethanol as a metabolic product. The cell may naturally comprise the required metabolic enzyme system or the cell may have been genetically modified to that purpose, e.g. as described by the review by Van Maris, the references cited therein, or as described in the present disclosure. Preferably, the fermentable carbohydrates in the hydrolysate comprise at least one carbohydrate selected from the group of hexoses, pentoses and oligosaccharides comprising one or more hexose and/or pentose units. In particular in case the recombinant cell is from the group Saccharomyces, preferably S. cerevisiae at least one carbohydrate selected from glucose, fructose, sucrose, maltose, galactose, xylose, arabinose and mannose is used. Good results have been obtained with glucose.

A method according to the invention is in particular suitable for preparing ethanol using a hydrolysate of at least one polymer selected from cellulose, hemicellulose and pectin, preferably at least one polymer selected from hemicellulose and pectin, because upon hydrolysis of these polymers acetate is typically released by hydrolysis or formed as a breakdown product. In particular, the hydrolysate may be hydrolysed lignocellulosic material, such as lignocellulosic biomass. For instance, lignocellulosic material may be selected from agricultural lignocellulosic material, for instance cotton, straw, (elephant) grass, bagasse, corn stover, lignocellulosic aquatic plant material, sugar beet pulp, citrus peels, lignocellulosic materials from forestry, such as lignocellulosic waste-materials from trees or bushes (trimmed/lopped/pruned plant material, saw dust, etc) or trees or bushes specifically grown as a source for lignocellulosic materials, e.g. poplar trees, (ligno) cellulosic waste from industry, e.g. wood-pulp, waste-paper.

Preferably, a lignocellulosic hydrolysate comprises one or more fermentable sugars (notably glucose, xylose and arabinose) and acetate, which have formed during hydrolysis. Thus, in a preferred embodiment of the invention the preparation of ethanol comprises a step wherein a lignocellulosic material is hydrolysed, thereby forming a hydrolysate comprising one or more fermentable carbohydrates, in particular glucose and optionally one or more other hexoses and pentoses, and acetate, which hydrolysate is thereafter contacted with a recombinant cell of the invention. The relative concentrations of acetate and fermentable carbohydrates in the substrate that is contacted with the recombinant cell of the invention can be modified by optimizing conditions for hydrolysis, by blending different hydrolysates and/or by blending with (partially) refined sources of carbohydrates and/or acetic acid.

Suitable hydrolysis methodology may be based on the Van Maris review cited above or the references cited therein, and include enzymatic hydrolysis, thermal hydrolysis, chemical hydrolysis and combinations thereof. The polymers are usually hydrolysed to the extent that at least 50%, preferably at least 90%, in particular at least 95% of the chains are degraded to monosaccharide units or to monosaccharide units and disaccharide units.

The invention is now illustrated by the following example:

### EXAMPLES

### EXAMPLE 1

### Materials and Methods

### Strain Construction and Maintenance

The Saccharomyces cerevisiae strains used (Table 1) originate from the CEN.PK family, which was previously identified as a suitable background for combined genetic and physiological studies (van Dijken et al. (2000) Enzyme Microb. Technol. 26:706-714).

TABLE 1
Saccharomyces cerevisiae strains used

| Strain | Relevant genotype | Source/reference |
|---|---|---|
| CEN.PK113-5D | MATa ura3 GPD1 GPD2 | EUROSCARF |
| | | strain collection, |
| | | Frankfurt, |
| | | Germany |
| IME076 | MATa ura3 GPD1 GPD2 | |
| (reference) | p426_GPD(URA3) | |
| CEN.PK102-3A | MATa ura3 leu2 GPD1 GPD2 | EUROSCARF |
| | | strain collection, |
| | | Frankfurt, |
| | | Germany |
| RWB0094 | MATa ura3 leu2 | BIRD |
| | gpd1(−1.1133)::loxP- | Engineering, |
| | KanMY-loxP | Rotterdam |
| | gpd2(−2.1281)::hphMX4 | |
| IMZ008 | MATa ura3 leu2 | |
| | gpd1(−1.1133)::loxP | |
| | gpd2(−2.1281)::hphMX4 | |
| | YEplac181(LEU2) | |
| IMZ132 | MATa ura3 leu2 | Deposited at |
| (CBS125049) | gpd1(−1.1133)::loxP | Centraalbureauvoor |
| | gpd2(−2.1281)::hphMX4 | Schimmelcultures |
| | YEplac181(LEU2) | on Jul. 16, 2009 |
| | pUDE43(URA3 pTHD3::mhpF | |
| | (E. coli)::CYC1t) | |
| IMZ127 | MATa ura3 leu2 | |
| | gpd1(−1.1133)::loxP | |
| | gpd2Δ(−2.1281)::hphMX4 | |
| | YEplac181(LEU2) | |
| | p426_GPD(URA3) | |

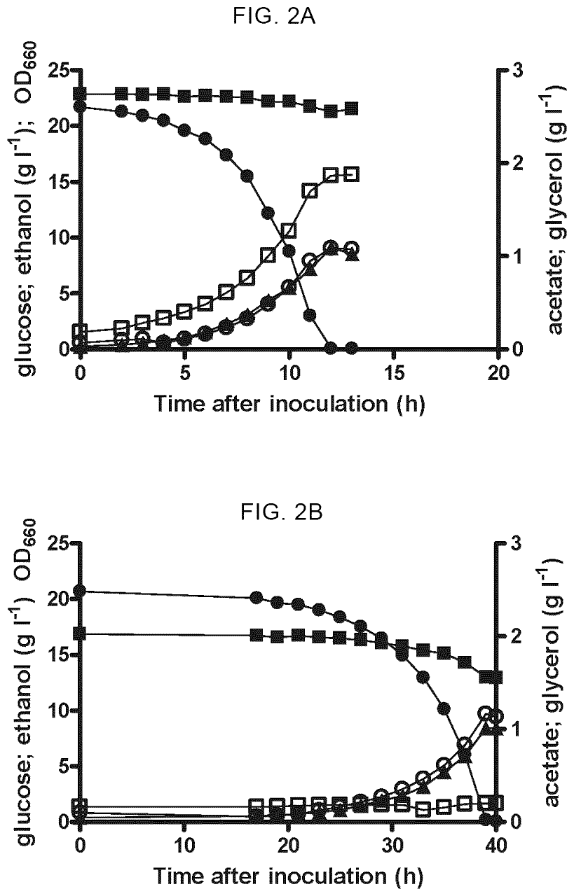

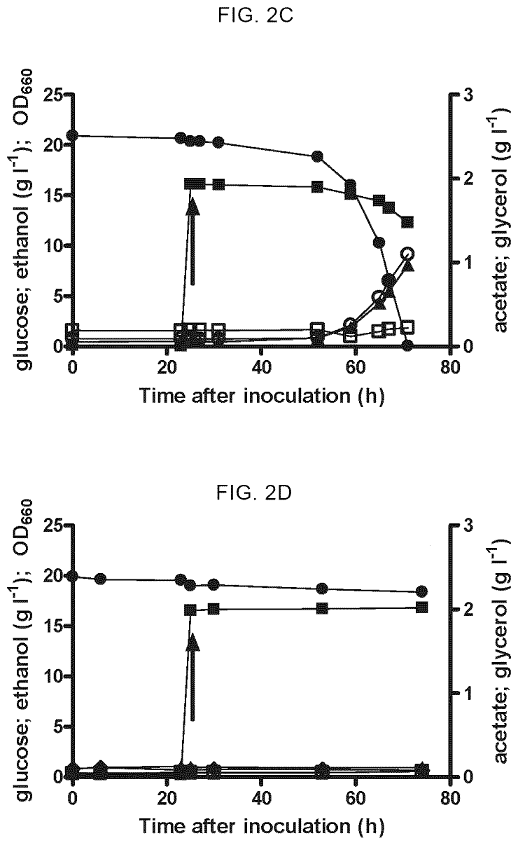

To disrupt the GPD1 gene (YDL022W), the loxP-KanMX-loxP disruption cassette may be amplified by PCR according to Güldener et al. (1996, Nucleic Acids Res. 24:2519-2524), using a primer set containing 45-nucleotide flanking regions homologous to sequences within the GPD1 gene and approximately 20-nucleotide homologous to sequences of the disruption module of pUG6 (Güldener et al. (1996) Nucleic Acids Res. 24:2519-2524) (FIG. 2, Table 2). Similarly, plasmid pAG32 ((Goldstein and McCusker (1999) Yeast 15:1541-1553) may be used as a template for PCR amplification of the hphMX4 disruption module. For construction of a GPD2 (YOL059W) disruption cassette a primer set may be used containing 45-nucleotide flanking regions homologous to sequences within the GPD2 gene and 20-nucleic acid sequences homologous to sequences of the disruption module of pAG32 (Table 2).

TABLE 2
Oligonucleotides for inactivation of the GPD1 and GPD2 genes
and for verification of correct disruption by diagnostic
PCR. Gene disruption oligonucleotides; nucleotides
homologous to the sequence to either the left (5′ side) or
right (3′ side) of the genes to be deleted are indicated in
capitals; lower case letters indicate nucleotides homologous
to the sequence of the disruption cassettes.

| Target gene | GPD1/YDL022W | GPD2/YOL059W |
|---|---|---|
| Gene disruption | fw | fw |
| primers | 5′TTGTACACCCCCCCCCTCC | 5′TCAATTCTCTTTCCCTTTCC |
| | ACAAACACAAATATTGATAAT | TTTTCCTTCGCTCCCCTTCCTT |
| | ATAAAcagctgaagcttcgta | ATCccaggctgaagcttcgtac |
| | cgc [SEQ ID NO: 5] | g [SEQ ID NO: 7] |
| | rv | rv |
| | 5′AATCTAATCTTCATGTAGA | 5′GTGTCTATTCGTCATCGATG |
| | TCTAATTCTTCAATCATGTCC | TCTAGCTCTTCAATCATCTCCG |
| | GGCGgcataggccactagtgg | GTAGgcataggccactagtgga |
| | atctg [SEQ ID NO: 6] | tc [SEQ ID NO: 8] |
| Verification | fw 5′ GPD1 | fw 5′ GPD2 |
| primers - | 5′CCCACCCACACCACCAATA | 5′ GTTCAGCAGCTCTTCTCTA |
| target gene | C [SEQ ID NO: 9] | C [SEQ ID NO: 11] |
| specific | rv 3′ GPD1 | rv 3′ GPD2 |
| | 5′CGGACGCCAGATGCTAGAA | 5′CCAAATGCGACATGAGTCAC |
| | G [SEQ ID NO: 10] | [SEQ ID NO: 12] |
| Verification | fw KANB | Fw KANB |
| primers - | 5′CGCACGTCAAGACTGTCAA | 5′CGCACGTCAAGACTGTCAAG |
| disruption | G [SEQ ID NO: 13) | [SEQ ID NO: 15] |
| cassette | rv KANA | rv KANA |
| specific | 5′TCGTATGTGAATGCTGGTC | 5′TCGTATGTGAATGCTGGTCG |
| | G [SEQ ID NO: 14] | [SEQ ID NO: 16] |

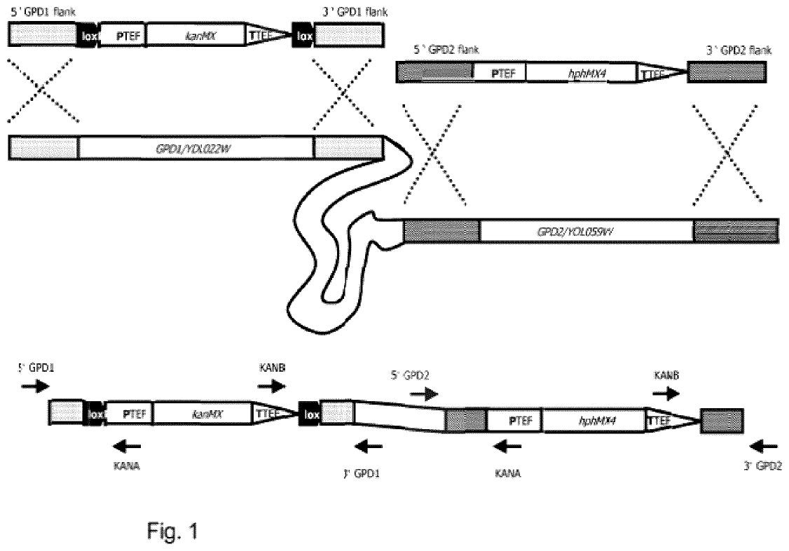

Transformation of the PCR amplified GPD1 and GPD2 disruption cassettes to Saccharomyces cerevisiae strain CEN.PK102-3A (Table 1) may be performed according to the protocols described by Güldener et al (Nucleic Acids Res. (1996) 24:2519-2524), followed by selection of transformants on YPD complex medium (Burke et al. (2000) Methods in yeast genetics. Cold Spring Harbour Press Plainview, NY) with 200 mg/L G-418 for strains transformed with the KanMX disruption cassette and 300 mg/L hygromycin B for strains transformed with the hphMX4 disruption cassette. Confirmation of correct integration of the GPD1 gene disruption cassette may be checked by colony PCR, using combinations of primer sets 5′ GPD1\KANA and 3′GPD1\KANB (Table 2). The correct inactivation of GPD2 can be similarly verified with primer sets 5′ GPD2\KANA and 3′GPD2\KANB (Table 2). Strain RWB0094, carrying deletions in the open reading frames of the GPD1 and GPD2 genes of strain CEN.PK102-3A (MATa ura3 leu2) were replaced by the loxP-KanMX-loxP cassette and the hphMX4 cassette, respectively, was acquired from BIRD Engineering, Rotterdam, The Netherlands. The KanMX marker of strain RWB0094 was removed by expression of the Cre recombinase (Guldener et al (1996) Nucleic Acids Res. 24:2519-2524) and its leucine auxotrophy was complemented by transformation with the LEU2-bearing plasmid YEPlac181 (Gietz, R. D. and S. Akio. (1988) Gene 74:527-534.), yielding strain IMZ008. FIG. 1 2 schematically shows the gene disruption procedure, replacement of the GPD1 ORF by KanMX and GPD2 ORF by hphMX4. Arrows indicate oligonucleotide primers for verification by diagnostic PCR of correct gene inactivation.

Transformation of strain IMZ008 with the URA3-bearing mhpF expression plasmid pUDE43 (see below) yielded the prototrophic, mhpF-expressing strain IMZ132, transformation with the URA3-bearing ‘empty’ vector p426_GPD yielded strain IMZ127. Finally, transformation of strain CEN.PK113-5D (ura3) with p426_GPD yielded the prototrophic GPD1 GPD2 reference strain IME076. Cultures transformed with deletion cassettes were plated on YPD complex medium containing G418 (200 mg l⁻¹) or hygromycin (200 mg l⁻¹). Successful integration of the deletion cassettes was confirmed by diagnostic PCR.

Stock cultures of all strains were grown in shake flasks containing 100 ml of synthetic medium (see below) with 20 g l⁻¹ glucose as the carbon source. After adding 30% (v/v) glycerol, 1-ml aliquots of stationary phase cultures were stored at −80° C.

### Plasmid Construction

The E. coli mhpF gene (EMBL accession number Y09555.7) was PCR amplified from E. coli K12 strain JM109 genomic DNA using primer pairs mhpF-FW (5′-GGGGACAAGTTTGTACAAAAAAGCAGGCTATGAGTAAGCGTAAAGTCGCCATTATCGG-3′ [SEQ ID NO:3]) and mhpF-RV (5′-GGGGACCACTTTGTACAAGAAAGCTGGGTGTTCATGCCGCTTCTCCTGCCTTGC-3′, [SEQ ID NO:4]), which contained attB1 and attB2 sequences, respectively. The polymerase chain reaction (PCR) was performed using Phusion® Hot Start High-Fidelity DNA Polymerase (Finnzymes Oy, Espoo, Finland) according to manufacturer specifications and in a Biometra TGradient Thermocycler (Biometra, Göttingen, Germany) with the following settings: 25 cycles of 10 s denaturation at 98° C. and 30 s annealing and extension at 72° C. The 1011 bp PCR product was cloned using Gateway® cloning technology (Invitrogen, Carlsbad, CA, USA). Plasmid pDONR221, using the BP reaction, was used to create the entry clone, designated as plasmid pUD64. From this entry clone and the multicopy plasmid pAG426GPD-ccdB (Addgene, Cambridge, MA, USA) the yeast expression plasmid pUDE43, was constructed employing the LR reaction. Transformations of recombination reaction products into competent E. coli K12 strain JM109 were performed according to the Z-Competent™ E. coli Transformation Kit (Zymoresearch Corporation, Orange, USA) and plated on LB media containing either ampicillin (100 mg l⁻¹) or kanamycin (50 mg·l⁻¹). Yeast transformations were performed according to Burke et al. (Methods in yeast genetics (2000.) Cold Spring Harbor Laboratory Press Plainview, NY.) After transformations with the yeast expression plasmid, cells were plated on synthetic media. Successful insertion of multicopy plasmid pUDE43 was confirmed by diagnostic PCR using the primer pairs for cloning.

### Cultivation and Media

Shake-flask cultivation was performed at 300C in a synthetic medium (46). The pH of the medium was adjusted to 6.0 with 2 M KOH prior to sterilization. Precultures were prepared by inoculating 100 ml medium containing 20 g l⁻¹ glucose in a 500 mL shake-flask with (1 ml) frozen stock culture. After 24 h incubation at 30° C. in an Innova® incubator shaker (200 rpm, New Brunswick Scientific, NJ, USA), cultures were transferred to bioreactors.

Anaerobic batch fermentations were carried out at 30° C. in 2-litre laboratory bioreactors (Applikon, Schiedam, The Netherlands) with a working volume of 1 litre. Synthetic medium with 20 g l⁻¹ glucose (46) was used for all fermentations and supplemented with 100 μl l⁻¹ of silicone antifoam (Silcolapse 5020, Caldic Belgium, Bleustar Silicones) as well as with the anaerobic growth factors, ergosterol (0.01 g l⁻¹) and Tween 80 (0.42 g l⁻¹) dissolved in ethanol. This resulted in 11-13 mM ethanol in the medium. Where indicated, acetic acid was added at a concentration of 2 g l⁻¹ and the pH was readjusted to 5.0 prior to inoculation. Culture pH was maintained at 5.0 by the automatic addition of 2M KOH. Cultures were stirred at 800 rpm and sparged with 0.5 L min⁻¹ nitrogen (<10 ppm oxygen). Dissolved oxygen was monitored with an autoclavable oxygen electrode (Applisens, Schiedam, The Netherlands). To minimize diffusion of oxygen, bioreactors were equipped with Norprene tubing (Cole Palmer Instrument Company, Vernon Hills, USA). All fermentations were carried out at least in duplicate.

### Determination of Culture Dry Weight and Optical Density

Culture samples (10 ml) at selected time intervals were filtered over pre-weighed nitrocellulose filters (pore size 0.45 μm; Gelman Laboratory, Ann Arbor, USA). After removal of medium the filters were washed with demineralized water and dried in a microwave oven (Bosch, Stuttgart, Germany) for 20 min at 350 W and weighed. Duplicate determinations varied by less than 1%. Culture growth was also monitored via optical density readings at a wavelength of 660 nm on a Novaspec® II spectrophotometer.

### Gas Analysis

Exhaust gas was cooled in a condensor (2° C.) and dried with a Permapure dryer type MD-110-48P-4 (Permapure, Toms River, USA). Oxygen and carbon dioxide concentrations were determined with a NGA 2000 analyzer (Rosemount Analytical, Orrville, USA). Exhaust gas-flow rate and carbon dioxide production rates were determined as described previously (3). In calculating these biomass-specific rates, a correction was made for volume changes caused by withdrawing culture samples.

### Enzyme Activity Assays

Cell extracts for activity assays of NAD⁺-dependent acetaldehyde dehydrogenase (acetylating) were prepared from exponentially growing anaerobic batch cultures as described previously (Abbott et al., Appl. Environ. Microbiol. 75:2320-2325). NAD⁺-dependent acetaldehyde dehydrogenase (acetylating) activity was measured at 30° C. by monitoring the oxidation of NADH at 340 nm. The reaction mixture (total volume 1 ml) contained 50 mM potassium phosphate buffer (pH 7.5), 15 mM NADH and cell extract. The reaction was started by addition of 0.5 mM acetyl-Coenzyme A. For glycerol 3-phosphate dehydrogenase (EC 1.1.1.8) activity determination, cell extracts were prepared as described above except that the phosphate buffer was replaced by triethanolamine buffer (10 mM, pH 5) (5,19). Glycerol-3-phosphate dehydrogenase activities were assayed in cell extracts at 30° C. as described previously (Blomberg and Adler (1989), J. Bacteriol. 171:1087-1092. Reaction rates were proportional to the amounts of cell extract added. Protein concentrations were determined by the Lowry method (Lowry et al (1951) J. Biol. Chem. 193:265-275) using bovine serum albumin as a standard.

### Results

### Growth and Product Formation in Anaerobic Batch Cultures

When cultures of the prototropic reference strain S. cerevisiae IME076 (GPD1 GPD2) were supplemented with 2.0 g l⁻¹ acetic acid, the specific growth rate (0.32 h⁻¹) was identical to that reported for cultures grown in the absence of acetic acid (0.34 h⁻¹), Kuyper et al. (2005) FEMS Yeast Res. 5:399-409. The addition of acetic acid led to a slight decrease of the biomass yield and, consequently a decrease of the glycerol yield on glucose relative cultures grown in the absence of acetic acid (FIG. 2, Table 3).

This effect has been attributed to the higher rate of glucose dissimilation for intracellular pH homeostasis due to diffusion of acetic acid into the cell, which in turn results in a lower biomass yield on glucose. Under the same conditions, an isogenic gpd1Δ gpd2Δ strain, in which absence of NAD⁺-dependent glycerol-3-phosphate dehydrogenase activity was confirmed in cell extracts (Table 2), was completely unable to grow anaerobically (FIG. 2), consistent with the notion that glycerol production via Gpd1 and Gpd2 is essential for NADH reoxidation in anaerobic cultures of S. cerevisiae.

TABLE 3
Physiology of the engineered S. cerevisiae strain IMZ132 and the
empty-vector reference strain IME076 during anaerobic batch cultivation
on synthetic medium (pH 5) with glucose-acetate mixtures

| Yeast strain | IME076 | IMZ132 |
|---|---|---|
| Relevant genotype | GPD1 GPD2 | gpd1Δ gpd2Δ + |
| | | mhpF |
| Glycerol 3-phosphate | 0.034 ± 0.003 | <0.002 |
| dehydrogenase | | |
| (μmol mg protein⁻¹ min⁻¹) | | |
| Acetaldehyde dehydrogenase | <0.002 | 0.020 ± 0.004 |
| (acetylating) | | |
| (μmol mg protein⁻¹ min⁻¹) | | |
| Specific growth rate (h⁻¹) | 0.32 ± 0.01 | 0.14 ± 0.01 |
| Biomass yield on glucose (g g⁻¹) | 0.083 ± 0.000 | 0.082 ± 0.009 |
| Biomass yield on acetate (g g⁻¹) | n.a. | 3.8 ± 0.5 |
| Glycerol yield on glucose (g g⁻¹) | 0.073 ± 0.007 | <0.002 |
| Ethanol yield on glucose (g g⁻¹) | 0.39 ± 0.01 | 0.43 ± 0.01 |
| Not corrected for evaporation | | |
| Ethanol yield on glucose (g g⁻¹) | 0.41 ± 0.01 | 0.47 ± 0.01 |
| Corrected for evaporation | | |

n.a., not applicable.

Expression of the E. coli mhpF gene in a gpd1Δ gpd2Δ strain, resulting in acetyl-CoA dependent rates of NADH reduction in cell extracts of 0.020 μmol min⁻¹ (mg protein)⁻¹ (Table 3), did not enable anaerobic growth when glucose was the sole carbon source. However, when the medium was supplemented with 2.0 g l⁻¹ acetic acid, exponential growth was observed at a specific growth rate of 0.14 h⁻¹. No formation of glycerol occurred during cultivation. (FIG. 2, Table 3). The trace amounts (<1 mM) of glycerol present in cultures of gpd1Δ gpd2Δ strains originate from the inoculum cultures which were started from frozen glycerol stocks. Ethanol was the major organic product and the small amounts of succinate and lactate produced were similar to those observed in cultures of the reference strain grown under the same conditions (data not shown).

It is observed that the IMZ132 fermentations (40 h) lasted longer than the wild type strain (15 h) and the anaerobic batch cultures were sparged with nitrogen gas. Accordingly, the fraction of ethanol lost through evaporation was higher for strain IMZ132. After determination of the kinetics of ethanol evaporation in sterile control experiments and correction of the ethanol yields, a 13% higher apparent ethanol yield on glucose was shown for the engineered strain according to the invention, using the linear pathway for NADH dependent reduction of acetic acid to ethanol (Table 3).

### Discussion

The present study provides a proof of principle that, stoichiometrically, the role of glycerol as a redox sink for anaerobic growth of S. cerevisiae can be fully replaced by a linear pathway for NADH-dependent reduction of acetate to ethanol. This offers interesting perspectives for large-scale ethanol production from feedstocks that contain acetic acid, such as lignocellulosic hydrolysates.

In addition to reducing the organic carbon content of spent media and increasing the ethanol yield, the reduction of acetic acid to ethanol may at least partially alleviate acetate inhibition of yeast growth and metabolism, which is especially problematic at low pH and during the consumption of pentose sugars by engineered yeast strains.

### Example 2

This example relates to the fermentative glycerol-free ethanol production by a gpd1Δ gpd2Δ Saccharomyce S. cerevisiae strain expressing a codon optimised version (SEQ ID NO:28) of the wild type dmpF gene from Pseudomonas sp. CF600 (GenBank No: CAA43226) (Shingler et al. (1992) J. Bacteriol. 174:71 1-24).

For this purpose, the same procedures and techniques used for the construction and evaluation of the performance of the strain IMZ132 (gpd1Δ gpd2Δ mhpF) were used. These procedures and techniques are described in Materials and Methods section of Example 1. In this work, transformation of strain IMZ008 (gpd1Δ gpd2Δura3Δ) with the URA3-bearing dmpF expression plasmid pUDE47 yielded the prototrophic, dmpF-expressing strain IMZ130. For the construction of plasmid pUDE47, a codon optimized copy of the dmpF gene (EMBL accession number X60835.1) from Pseudomonas sp. CF600 was ligated into p426_GPD plasmid. Codon optimization for the expression in S. cerevisiae and ligation into the plasmid p426_GPD were performed by BaseClear BV (Leiden, The Netherlands). Successful insertion of multicopy plasmid pUDE47 was confirmed using diagnostic colony PCR using the primer pairs dmpF-FW (CATTGATTGCGCCATACG (SEQ ID NO:30)) and dmpF-RV (CCGGTAATATCGGAACAGAC (SEQ ID NO:31).

### Results

### Growth and Product Formation in Anaerobic Batch Cultures

Expression of the Pseudomonas sp. CF600 dmpF gene in a gpd1Δ gpd2Δ S. cerevisiae strain gave similar results to the obtained for the expression of E. coli mhpF gene in the same strain. In anaerobic batch fermentations, similar to strain IMZ132 (gpd1Δ gpd2Δ mhpF), functional expression of dmpF gene together with supplementation of the medium with 2.0 g l⁻¹ acetic acid, resulted in exponential growth with a specific growth rate of 0.11 h⁻¹ (FIG. 1). Strain IMZ130 (gpd1 [symbol] gpd2 [symbol] dmpF) had a slightly longer batch time (55 h) than strain IMZ132 (40 h). During cultivation, initial concentration of 20 g l⁻¹ of glucose was completely consumed, while no glycerol formation was observed. At the same time, acetate was consumed from 2.1 g l⁻¹ initial concentration to 1.6 g l⁻¹ final concentration. Ethanol was the major organic product, exhibiting an ethanol yield on glucose of 0.48 g g⁻¹ (yield corrected by ethanol evaporation). Small amounts of succinate and lactate were produced, similar to those observed in cultures of the reference strain grown under the same conditions.

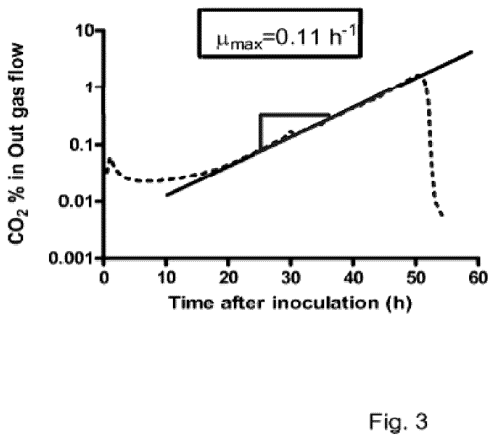

FIG. 3 shows the volumetric CO2 percentage present in the out flow of a batch fermentation inoculated with strain IMZ130 (gpd1Δ gpd2Δ dmpF). The graph is presented in a logarithmic scale on the y-axis in order to demonstrate exponential growth and calculate the maximum specific growth rate.

The insertion of a synthetic codon optimized copy of the gene dmpF from Pseudomonas sp. CF600, provides another example that it is stoichiometrically possible to substitute glycerol formation as a redox sink in anaerobic growth of S. cerevisiae by a linear metabolic pathway for NADH-dependent reduction of acetate to ethanol. Also this example shows that the insertion of (acetalyting) acetaldehyde dehydrogenase in a gpd1Δ gpd2Δ S. cerevisiae strain resulted in higher ethanol yields on glucose, no formation of by-product glycerol, and the consumption of fermentation inhibitor compound acetate.

E. coli mhpF gene Acetaldehyde dehydrogenase, acylating
SEQ ID NO: 1
ATGAGTAAGCGTAAAGTCGCCATTATCGGTTCTGGCAACATTGGTACCGATCTGATGATTAAA
ATTTTGCGTCACGGTCAGCATCTGGAGATGGCGGTGATGGTTGGCATTGATCCTCAGTCCGAC
GGTCTGGCGCGCGCCAGACGTATGGGCGTCGCCACCACCCATGAAGGGGTGATCGGACTGATG
AACATGCCTGAATTTGCTGATATCGACATTGTATTTGATGCGACCAGCGCCGGTGCTCATGTG
AAAAACGATGCCGCTTTACGCGAAGCGAAACCGGATATTCGCTTAATTGACCTGACGCCTGCT
GCCATCGGCCCTTACTGCGTGCCGGTGGTTAACCTCGAGGCGAACGTCGATCAACTGAACGTC
AACATGGTCACCTGCGGCGGCCAGGCCACCATTCCAATGGTGGCGGCAGTTTCACGCGTGGCG
CGTGTTCATTACGCCGAAATTATCGCTTCTATCGCCAGTAAATCTGCCGGACCTGGCACGCGT
GCCAATATCGATGAATTTACGGAAACCACTTCCCGAGCCATTGAAGTGGTGGGCGGCGCGGCA
AAAGGGAAGGCGATTATTGTGCTTAACCCAGCAGAGCCACCGTTGATGATGCGTGACACGGTG
TATGTATTGAGCGACGAAGCTTCACAAGATGATATCGAAGCCTCAATCAATGAAATGGCTGAG
GCGGTGCAGGCTTACGTACCGGGTTATCGCCTGAAACAGCGCGTGCAGTTTGAAGTTATCCCG
CAGGATAAACCGGTCAATTTACCGGGCGTGGGGCAATTCTCCGGACTGAAAACAGCGGTCTGG
CTGGAAGTCGAAGGCGCAGCGCATTATCTGCCTGCCTATGCGGGCAACCTCGACATTATGACT
TCCAGTGCGCTGGCGACAGCGGAAAAAATGGCCCAGTCACTGGCGCGCAAGGCAGGAGAAGCG
GCATGA
E. coli Acetaldehyde dehydrogenase OS = Escherichia coli
(strain K12) GN = mhpF PE = 1 SV = 1
SEQ ID NO: 2
MSKRKVAIIGSGNIGTDLMIKILRHGQHLEMAVMVGIDPQSDGLARARRMGVATTHEGVIGLM
NMPEFADIDIVFDATSAGAHVKNDAALREAKPDIRLIDLTPAAIGPYCVPVVNLEANVDQLNV
NMVTCGGQATIPMVAAVSRVARVHYAEIIASIASKSAGPGTRANIDEFTETTSRAIEVVGGAA
KGKAIIVLNPAEPPLMMRDTVYVLSDEASQDDIEASINEMAEAVQAYVPGYRLKQRVQFEVIP
QDKPVNLPGVGQFSGLKTAVVVLEVEGAAHYLPAYAGNLDIMTSSALATAEKMAQSLARKAGE
AA
primer mhpF-FW
SEQ ID NO: 3
GGGGACAAGTTTGTACAAAAAAGCAGGCTATGAGTAAGCGTAAAGTCGCCATTATCGG
primer mhpF-RV
SEQ ID NO: 4
GGGGACCACTTTGTACAAGAAAGCTGGGTGTTCATGCCGCTTCTCCTGCCTTGC
GPD1/YDL022W fw gene disruption primer
SEQ ID NO: 5
TTGTACACCCCCCCCCTCCACAAACACAAATATTGATAATATAAAcagctgaagcttcgtacg
c
GPD1/YDL022W rv gene disruption primer
SEQ ID NO: 6
AATCTAATCTTCATGTAGATCTAATTCTTCAATCATGTCCGGCGgcataggccactagtggat
ctg
GPD2/YOL059W fw gene disruption primer
SEQ ID NO: 7
TCAATTCTCTTTCCCTTTCCTTTTCCTTCGCTCCCCTTCCTTATCccaggctgaagcttcgta
cg
GPD2/YOL059W rv gene disruption primer
SEQ ID NO: 8
GTGTCTATTCGTCATCGATGTCTAGCTCTTCAATCATCTCCGGTAGgcataggccactagtgg
atc
GPD1/YDL022W fw verification primer
SEQ ID NO: 9
CCCACCCACACCACCAATAC
GPD1/YDL022W rv verification primer
SEQ ID NO: 10
CGGACGCCAGATGCTAGAAG
GPD2/YOL059W fw verification primer
SEQ ID NO: 11
GTTCAGCAGCTCTTCTCTAC
GPD2/YOL059W rv verification primer
SEQ ID NO: 12
CCAAATGCGACATGAGTCAC
GPD1/YDL022W fw disruption cassette specific verification primer
SEQ ID NO: 13
CGCACGTCAAGACTGTCAAG
GPD1/YDL022W rv disruption cassette specific verification primer
SEQ ID NO: 14
TCGTATGTGAATGCTGGTCG
GPD2/YOL059W fw disruption cassette specific verification primer
SEQ ID NO: 15
CGCACGTCAAGACTGTCAAG
GPD2/YOL059W rv disruption cassette specific verification primer
SEQ ID NO: 16
TCGTATGTGAATGCTGGTCG
S. cerevisiae ACS 1 gene
SEQ ID NO: 17
ATGTCGCCCTCTGCCGTACAATCATCAAAACTAGAAGAACAGTCAAGTGAAATTGACAAGTTG
AAAGCAAAAATGTCCCAGTCTGCCGCCACTGCGCAGCAGAAGAAGGAACATGAGTATGAACAT
TTGACTTCGGTCAAGATCGTGCCACAACGGCCCATCTCAGATAGACTGCAGCCCGCAATTGCT
ACCCACTATTCTCCACACTTGGACGGGTTGCAGGACTATCAGCGCTTGCACAAGGAGTCTATT
GAAGACCCTGCTAAGTTCTTCGGTTCTAAAGCTACCCAATTTTTAAACTGGTCTAAGCCATTC
GATAAGGTGTTCATCCCAGACCCTAAAACGGGCAGGCCCTCCTTCCAGAACAATGCATGGTTC
CTCAACGGCCAATTAAACGCCTGTTACAACTGTGTTGACAGACATGCCTTGAAGACTCCTAAC
AAGAAAGCCATTATTTTCGAAGGTGACGAGCCTGGCCAAGGCTATTCCATTACCTACAAGGAA
CTACTTGAAGAAGTTTGTCAAGTGGCACAAGTGCTGACTTACTCTATGGGCGTTCGCAAGGGC
GATACTGTTGCCGTGTACATGCCTATGGTCCCAGAAGCAATCATAACCTTGTTGGCCATTTCC
CGTATCGGTGCCATTCACTCCGTAGTCTTTGCCGGGTTTTCTTCCAACTCCTTGAGAGATCGT
ATCAACGATGGGGACTCTAAAGTTGTCATCACTACAGATGAATCCAACAGAGGTGGTAAAGTC
ATTGAGACTAAAAGAATTGTTGATGACGCGCTAAGAGAGACCCCAGGCGTGAGACACGTCTTG
GTTTATAGAAAGACCAACAATCCATCTGTTGCTTTCCATGCCCCCAGAGATTTGGATTGGGCA
ACAGAAAAGAAGAAATACAAGACCTACTATCCATGCACACCCGTTGATTCTGAGGATCCATTA
TTCTTGTTGTATACGTCTGGTTCTACTGGTGCCCCCAAGGGTGTTCAACATTCTACCGCAGGT
TACTTGCTGGGAGCTTTGTTGACCATGCGCTACACTTTTGACACTCACCAAGAAGACGTTTTC
TTCACAGCTGGAGACATTGGCTGGATTACAGGCCACACTTATGTGGTTTATGGTCCCTTACTA
TATGGTTGTGCCACTTTGGTCTTTGAAGGGACTCCTGCGTACCCAAATTACTCCCGTTATTGG
GATATTATTGATGAACACAAAGTCACCCAATTTTATGTTGCGCCAACTGCTTTGCGTTTGTTG
AAAAGAGCTGGTGATTCCTACATCGAAAATCATTCCTTAAAATCTTTGCGTTGCTTGGGTTCG
GTCGGTGAGCCAATTGCTGCTGAAGTTTGGGAGTGGTACTCTGAAAAAATAGGTAAAAATGAA
ATCCCCATTGTAGACACCTACTGGCAAACAGAATCTGGTTCGCATCTGGTCACCCCGCTGGCT
GGTGGTGTTACACCAATGAAACCGGGTTCTGCCTCATTCCCCTTCTTCGGTATTGATGCAGTT
GTTCTTGACCCTAACACTGGTGAAGAACTTAACACCAGCCACGCAGAGGGTGTCCTTGCCGTC
AAAGCTGCATGGCCATCATTTGCAAGAACTATTTGGAAAAATCATGATAGGTATCTAGACACT
TATTTGAACCCTTACCCTGGCTACTATTTCACTGGTGATGGTGCTGCAAAGGATAAGGATGGT
TATATCTGGATTTTGGGTCGTGTAGACGATGTGGTGAACGTCTCTGGTCACCGTCTGTCTACC
GCTGAAATTGAGGCTGCTATTATCGAAGATCCAATTGTGGCCGAGTGTGCTGTTGTCGGATTC
AACGATGACTTGACTGGTCAAGCAGTTGCTGCATTTGTGGTGTTGAAAAACAAATCTAGTTGG
TCCACCGCAACAGATGATGAATTACAAGATATCAAGAAGCATTTGGTCTTTACTGTTAGAAAA
GACATCGGGCCATTTGCCGCACCAAAATTGATCATTTTAGTGGATGACTTGCCCAAGACAAGA
TCCGGCAAAATTATGAGACGTATTTTAAGAAAAATCCTAGCAGGAGAAAGTGACCAACTAGGC
GACGTTTCTACATTGTCAAACCCTGGCATTGTTAGACATCTAATTGATTCGGTCAAGTTGTAA
S. cerevisiae ACS 2 gene
SEQ ID NO: 18
ATGACAATCAAGGAACATAAAGTAGTTTATGAAGCTCACAACGTAAAGGCTCTTAAGGCTCCT
CAACATTTTTACAACAGCCAACCCGGCAAGGGTTACGTTACTGATATGCAACATTATCAAGAA
ATGTATCAACAATCTATCAATGAGCCAGAAAAATTCTTTGATAAGATGGCTAAGGAATACTTG
CATTGGGATGCTCCATACACCAAAGTTCAATCTGGTTCATTGAACAATGGTGATGTTGCATGG
TTTTTGAACGGTAAATTGAATGCATCATACAATTGTGTTGACAGACATGCCTTTGCTAATCCC
GACAAGCCAGCTTTGATCTATGAAGCTGATGACGAATCCGACAACAAAATCATCACATTTGGT
GAATTACTCAGAAAAGTTTCCCAAATCGCTGGTGTCTTAAAAAGCTGGGGCGTTAAGAAAGGT
GACACAGTGGCTATCTATTTGCCAATGATTCCAGAAGCGGTCATTGCTATGTTGGCTGTGGCT
CGTATTGGTGCTATTCACTCTGTTGTCTTTGCTGGGTTCTCCGCTGGTTCGTTGAAAGATCGT
GTCGTTGACGCTAATTCTAAAGTGGTCATCACTTGTGATGAAGGTAAAAGAGGTGGTAAGACC
ATCAACACTAAAAAAATTGTTGACGAAGGTTTGAACGGAGTCGATTTGGTTTCCCGTATCTTG
GTTTTCCAAAGAACTGGTACTGAAGGTATTCCAATGAAGGCCGGTAGAGATTACTGGTGGCAT
GAGGAGGCCGCTAAGCAGAGAACTTACCTACCTCCTGTTTCATGTGACGCTGAAGATCCTCTA
TTTTTATTATACACTTCCGGTTCCACTGGTTCTCCAAAGGGTGTCGTTCACACTACAGGTGGT
TATTTATTAGGTGCCGCTTTAACAACTAGATACGTTTTTGATATTCACCCAGAAGATGTTCTC
TTCACTGCCGGTGACGTCGGCTGGATCACGGGTCACACCTATGCTCTATATGGTCCATTAACC
TTGGGTACCGCCTCAATAATTTTCGAATCCACTCCTGCCTACCCAGATTATGGTAGATATTGG
AGAATTATCCAACGTCACAAGGCTACCCATTTCTATGTGGCTCCAACTGCTTTAAGATTAATC
AAACGTGTAGGTGAAGCCGAAATTGCCAAATATGACACTTCCTCATTACGTGTCTTGGGTTCC
GTCGGTGAACCAATCTCTCCAGACTTATGGGAATGGTATCATGAAAAAGTGGGTAACAAAAAC
TGTGTCATTTGTGACACTATGTGGCAAACAGAGTCTGGTTCTCATTTAATTGCTCCTTTGGCA
GGTGCTGTCCCAACAAAACCTGGTTCTGCTACCGTGCCATTCTTTGGTATTAACGCTTGTATC
ATTGACCCTGTTACAGGTGTGGAATTAGAAGGTAATGATGTCGAAGGTGTCCTTGCCGTTAAA
TCACCATGGCCATCAATGGCTAGATCTGTTTGGAACCACCACGACCGTTACATGGATACTTAC
TTGAAACCTTATCCTGGTCACTATTTCACAGGTGATGGTGCTGGTAGAGATCATGATGGTTAC
TACTGGATCAGGGGTAGAGTTGACGACGTTGTAAATGTTTCCGGTCATAGATTATCCACATCA
GAAATTGAAGCATCTATCTCAAATCACGAAAACGTCTCGGAAGCTGCTGTTGTCGGTATTCCA
GATGAATTGACCGGTCAAACCGTCGTTGCATATGTTTCCCTAAAAGATGGTTATCTACAAAAC
AACGCTACTGAAGGTGATGCAGAACACATCACACCAGATAATTTACGTAGAGAATTGATCTTA
CAAGTTAGGGGTGAGATTGGTCCTTTCGCCTCACCAAAAACCATTATTCTAGTTAGAGATCTA
CCAAGAACAAGGTCAGGAAAGATTATGAGAAGAGTTCTAAGAAAGGTTGCTTCTAACGAAGCC
GAACAGCTAGGTGACCTAACTACTTTGGCCAACCCAGAAGTTGTACCTGCCATCATTTCTGCT
GTAGAGAACCAATTTTTCTCTCAAAAAAAGAAATAA
S. cerevisiae ADH1
SEQ ID NO: 19
ATGTCTATCCCAGAAACTCAAAAAGGTGTTATCTTCTACGAATCCCACGGTAAGTTGGAATAC
AAAGATATTCCAGTTCCAAAGCCAAAGGCCAACGAATTGTTGATCAACGTTAAATACTCTGGT
GTCTGTCACACTGACTTGCACGCTTGGCACGGTGACTGGCCATTGCCAGTTAAGCTACCATTA
GTCGGTGGTCACGAAGGTGCCGGTGTCGTTGTCGGCATGGGTGAAAACGTTAAGGGCTGGAAG
ATCGGTGACTACGCCGGTATCAAATGGTTGAACGGTTCTTGTATGGCCTGTGAATACTGTGAA
TTGGGTAACGAATCCAACTGTCCTCACGCTGACTTGTCTGGTTACACCCACGACGGTTCTTTC
CAACAATACGCTACCGCTGACGCTGTTCAAGCCGCTCACATTCCTCAAGGTACCGACTTGGCC
CAAGTCGCCCCCATCTTGTGTGCTGGTATCACCGTCTACAAGGCTTTGAAGTCTGCTAACTTG
ATGGCCGGTCACTGGGTTGCTATCTCCGGTGCTGCTGGTGGTCTAGGTTCTTTGGCTGTTCAA
TACGCCAAGGCTATGGGTTACAGAGTCTTGGGTATTGACGGTGGTGAAGGTAAGGAAGAATTA
TTCAGATCCATCGGTGGTGAAGTCTTCATTGACTTCACTAAGGAAAAGGACATTGTCGGTGCT
GTTCTAAAGGCCACTGACGGTGGTGCTCACGGTGTCATCAACGTTTCCGTTTCCGAAGCCGCT
ATTGAAGCTTCTACCAGATACGTTAGAGCTAACGGTACCACCGTTTTGGTCGGTATGCCAGCT
GGTGCCAAGTGTTGTTCTGATGTCTTCAACCAAGTCGTCAAGTCCATCTCTATTGTTGGTTCT
TACGTCGGTAACAGAGCTGACACCAGAGAAGCTTTGGACTTCTTCGCCAGAGGTTTGGTCAAG
TCTCCAATCAAGGTTGTCGGCTTGTCTACCTTGCCAGAAATTTACGAAAAGATGGAAAAGGGT
CAAATCGTTGGTAGATACGTTGTTGACACTTCTAAATAA
S. cerevisiae ADH2
SEQ ID NO: 20
ATGTCTATTCCAGAAACTCAAAAAGCCATTATCTTCTACGAATCCAACGGCAAGTTGGAGCAT
AAGGATATCCCAGTTCCAAAGCCAAAGCCCAACGAATTGTTAATCAACGTCAAGTACTCTGGT
GTCTGCCACACCGATTTGCACGCTTGGCATGGTGACTGGCCATTGCCAACTAAGTTACCATTA
GTTGGTGGTCACGAAGGTGCCGGTGTCGTTGTCGGCATGGGTGAAAACGTTAAGGGCTGGAAG
ATCGGTGACTACGCCGGTATCAAATGGTTGAACGGTTCTTGTATGGCCTGTGAATACTGTGAA
TTGGGTAACGAATCCAACTGTCCTCACGCTGACTTGTCTGGTTACACCCACGACGGTTCTTTC
CAAGAATACGCTACCGCTGACGCTGTTCAAGCCGCTCACATTCCTCAAGGTACTGACTTGGCT
GAAGTCGCGCCAATCTTGTGTGCTGGTATCACCGTATACAAGGCTTTGAAGTCTGCCAACTTG
AGAGCAGGCCACTGGGCGGCCATTTCTGGTGCTGCTGGTGGTCTAGGTTCTTTGGCTGTTCAA
TATGCTAAGGCGATGGGTTACAGAGTCTTAGGTATTGATGGTGGTCCAGGAAAGGAAGAATTG
TTTACCTCGCTCGGTGGTGAAGTATTCATCGACTTCACCAAAGAGAAGGACATTGTTAGCGCA
GTCGTTAAGGCTACCAACGGCGGTGCCCACGGTATCATCAATGTTTCCGTTTCCGAAGCCGCT
ATCGAAGCTTCTACCAGATACTGTAGGGCGAACGGTACTGTTGTCTTGGTTGGTTTGCCAGCC
GGTGCAAAGTGCTCCTCTGATGTCTTCAACCACGTTGTCAAGTCTATCTCCATTGTCGGCTCT
TACGTGGGGAACAGAGCTGATACCAGAGAAGCCTTAGATTTCTTTGCCAGAGGTCTAGTCAAG
TCTCCAATAAAGGTAGTTGGCTTATCCAGTTTACCAGAAATTTACGAAAAGATGGAGAAGGGC
CAAATTGCTGGTAGATACGTTGTTGACACTTCTAAATAA
S. cerevisiae ADH3
SEQ ID NO: 21
ATGTTGAGAACGTCAACATTGTTCACCAGGCGTGTCCAACCAAGCCTATTTTCTAGAAACATT
CTTAGATTGCAATCCACAGCTGCAATCCCTAAGACTCAAAAAGGTGTCATCTTTTATGAGAAT
AAGGGGAAGCTGCATTACAAAGATATCCCTGTCCCCGAGCCTAAGCCAAATGAAATTTTAATC
AACGTTAAATATTCTGGTGTATGTCACACCGATTTACATGCTTGGCACGGCGATTGGCCATTA
CCTGTTAAACTACCATTAGTAGGTGGTCATGAAGGTGCTGGTGTAGTTGTCAAACTAGGTTCC
AATGTCAAGGGCTGGAAAGTCGGTGATTTAGCAGGTATCAAATGGCTGAACGGTTCTTGTATG
ACATGCGAATTCTGTGAATCAGGTCATGAATCAAATTGTCCAGATGCTGATTTATCTGGTTAC
ACTCATGATGGTTCTTTCCAACAATTTGCGACCGCTGATGCTATTCAAGCCGCCAAAATTCAA
CAGGGTACCGACTTGGCCGAAGTAGCCCCAATATTATGTGCTGGTGTTACTGTATATAAAGCA
CTAAAAGAGGCAGACTTGAAAGCTGGTGACTGGGTTGCCATCTCTGGTGCTGCAGGTGGCTTG
GGTTCCTTGGCCGTTCAATATGCAACTGCGATGGGTTACAGAGTTCTAGGTATTGATGCAGGT
GAGGAAAAGGAAAAACTTTTCAAGAAATTGGGGGGTGAAGTATTCATCGACTTTACTAAAACA
AAGAATATGGTTTCTGACATTCAAGAAGCTACCAAAGGTGGCCCTCATGGTGTCATTAACGTT
TCCGTTTCTGAAGCCGCTATTTCTCTATCTACGGAATATGTTAGACCATGTGGTACCGTCGTT
TTGGTTGGTTTGCCCGCTAACGCCTACGTTAAATCAGAGGTATTCTCTCATGTGGTGAAGTCC
ATCAATATCAAGGGTTCTTATGTTGGTAACAGAGCTGATACGAGAGAAGCCTTAGACTTCTTT
AGCAGAGGTTTGATCAAATCACCAATCAAAATTGTTGGATTATCTGAATTACCAAAGGTTTAT
GACTTGATGGAAAAGGGCAAGATTTTGGGTAGATACGTCGTCGATACTAGTAAATAA
S. cerevisiae ADH4
SEQ ID NO: 22
ATGTCTTCCGTTACTGGGTTTTACATTCCACCAATCTCTTTCTTTGGTGAAGGTGCTTTAGAA
GAAACCGCTGATTACATCAAAAACAAGGATTACAAAAAGGCTTTGATCGTTACTGATCCTGGT
ATTGCAGCTATTGGTCTCTCCGGTAGAGTCCAAAAGATGTTGGAAGAACGTGACTTAAACGAG
TTGCTATCTATGACAAAACTCAACCAAACCCAAATATTGCCAATGTCACCTGGTTTGAAGGTT
TTGAAGGAACAAAACTCTGAAATTGTTGTTTCCATTGGTGGTGGTTCTGCTCACGACAATGCT
AAGGCCATTGCTTTATTGGCTACTAACGGTGGGGAAATCGGAGACTATGAAGGTGTCAATCAA
TCTAAGAAGGCTGCTTTACCACTATTTGCCATCAACACTACTGCTGGTACTGCTTCCGAAATG
ACCAGATTCACTATTATCTCTAATGAAGAAAAGAAAATCAAGATGGCTATCATTGACAACAAC
GTCACTCCAGCTGTTGCTGTCAACGATCCATCTACCATGTTTGGTTTGCCACCTGCTTTGACT
GCTGCTACTGGTCTAGATGCTTTGACTCACTGTATCGAAGCTTATGTTTCCACCGCCTCTAAC
CCAATCACCGATGCCTGTGCTTTGAAGGGTATTGATTTGATCAATGAAAGCTTAGTCGCTGCA
TACAAAGACGGTAAAGACAAGAAGGCCAGAACTGACATGTGTTACGCTGAATACTTGGCAGGT
ATGGCTTTCAACAATGCTTCTCTAGGTTATGTTCATGCCCTTGCTCATCAACTTGGTGGTTTC
TACCACTTGCCTCATGGTGTTTGTAACGCTGTCTTGTTGCCTCATGTTCAAGAGGCCAACATG
CAATGTCCAAAGGCCAAGAAGAGATTAGGTGAAATTGCTTTGCATTTCGGTGCTTCTCAAGAA
GATCCAGAAGAAACCATCAAGGCTTTGCACGTTTTAAACAGAACCATGAACATTCCAAGAAAC
TTGAAAGAATTAGGTGTTAAAACCGAAGATTTTGAAATTTTGGCTGAACACGCCATGCATGAT
GCCTGCCATTTGACTAACCCAGTTCAATTCACCAAAGAACAAGTGGTTGCCATTATCAAGAAA
GCCTATGAATATTAA
S. cerevisiae ADH5
SEQ ID NO: 23
ATGCCTTCGCAAGTCATTCCTGAAAAACAAAAGGCTATTGTCTTTTATGAGACAGATGGAAAA
TTGGAATATAAAGACGTCACAGTTCCGGAACCTAAGCCTAACGAAATTTTAGTCCACGTTAAA
TATTCTGGTGTTTGTCATAGTGACTTGCACGCGTGGCACGGTGATTGGCCATTTCAATTGAAA
TTTCCATTAATCGGTGGTCACGAAGGTGCTGGTGTTGTTGTTAAGTTGGGATCTAACGTTAAG
GGCTGGAAAGTCGGTGATTTTGCAGGTATAAAATGGTTGAATGGGACTTGCATGTCCTGTGAA
TATTGTGAAGTAGGTAATGAATCTCAATGTCCTTATTTGGATGGTACTGGCTTCACACATGAT
GGTACTTTTCAAGAATACGCAACTGCCGATGCCGTTCAAGCTGCCCATATTCCACCAAACGTC
AATCTTGCTGAAGTTGCCCCAATCTTGTGTGCAGGTATCACTGTTTATAAGGCGTTGAAAAGA
GCCAATGTGATACCAGGCCAATGGGTCACTATATCCGGTGCATGCGGTGGCTTGGGTTCTCTG
GCAATCCAATACGCCCTTGCTATGGGTTACAGGGTCATTGGTATCGATGGTGGTAATGCCAAG
CGAAAGTTATTTGAACAATTAGGCGGAGAAATATTCATCGATTTCACGGAAGAAAAAGACATT
GTTGGTGCTATAATAAAGGCCACTAATGGCGGTTCTCATGGAGTTATTAATGTGTCTGTTTCT
GAAGCAGCTATCGAGGCTTCTACGAGGTATTGTAGGCCCAATGGTACTGTCGTCCTGGTTGGT
ATGCCAGCTCATGCTTACTGCAATTCCGATGTTTTCAATCAAGTTGTAAAATCAATCTCCATC
GTTGGATCTTGTGTTGGAAATAGAGCTGATACAAGGGAGGCTTTAGATTTCTTCGCCAGAGGT
TTGATCAAATCTCCGATCCACTTAGCTGGCCTATCGGATGTTCCTGAAATTTTTGCAAAGATG
GAGAAGGGTGAAATTGTTGGTAGATATGTTGTTGAGACTTCTAAATGA
SEQ ID NO 24: S. cerevisiae GPD1
ATGTCTGCTGCTGCTGATAGATTAAACTTAACTTCCGGCCACTTGAATGCTGGTAGAAAGAGA
AGTTCCTCTTCTGTTTCTTTGAAGGCTGCCGAAAAGCCTTTCAAGGTTACTGTGATTGGATCT
GGTAACTGGGGTACTACTATTGCCAAGGTGGTTGCCGAAAATTGTAAGGGATACCCAGAAGTT
TTCGCTCCAATAGTACAAATGTGGGTGTTCGAAGAAGAGATCAATGGTGAAAAATTGACTGAA
ATCATAAATACTAGACATCAAAACGTGAAATACTTGCCTGGCATCACTCTACCCGACAATTTG
GTTGCTAATCCAGACTTGATTGATTCAGTCAAGGATGTCGACATCATCGTTTTCAACATTCCA
CATCAATTTTTGCCCCGTATCTGTAGCCAATTGAAAGGTCATGTTGATTCACACGTCAGAGCT
ATCTCCTGTCTAAAGGGTTTTGAAGTTGGTGCTAAAGGTGTCCAATTGCTATCCTCTTACATC
ACTGAGGAACTAGGTATTCAATGTGGTGCTCTATCTGGTGCTAACATTGCCACCGAAGTCGCT
CAAGAACACTGGTCTGAAACAACAGTTGCTTACCACATTCCAAAGGATTTCAGAGGCGAGGGC
AAGGACGTCGACCATAAGGTTCTAAAGGCCTTGTTCCACAGACCTTACTTCCACGTTAGTGTC
ATCGAAGATGTTGCTGGTATCTCCATCTGTGGTGCTTTGAAGAACGTTGTTGCCTTAGGTTGT
GGTTTCGTCGAAGGTCTAGGCTGGGGTAACAACGCTTCTGCTGCCATCCAAAGAGTCGGTTTG
GGTGAGATCATCAGATTCGGTCAAATGTTTTTCCCAGAATCTAGAGAAGAAACATACTACCAA
GAGTCTGCTGGTGTTGCTGATTTGATCACCACCTGCGCTGGTGGTAGAAACGTCAAGGTTGCT
AGGCTAATGGCTACTTCTGGTAAGGACGCCTGGGAATGTGAAAAGGAGTTGTTGAATGGCCAA
TCCGCTCAAGGTTTAATTACCTGCAAAGAAGTTCACGAATGGTTGGAAACATGTGGCTCTGTC
GAAGACTTCCCATTATTTGAAGCCGTATACCAAATCGTTTACAACAACTACCCAATGAAGAAC
CTGCCGGACATGATTGAAGAATTAGATCTACATGAAGATTAG
SEQ ID NO: 25: S. cerevisiae GPD2
ATGCTTGCTGTCAGAAGATTAACAAGATACACATTCCTTAAGCGAACGCATCCGGTGTTATAT
ACTCGTCGTGCATATAAAATTTTGCCTTCAAGATCTACTTTCCTAAGAAGATCATTATTACAA
ACACAACTGCACTCAAAGATGACTGCTCATACTAATATCAAACAGCACAAACACTGTCATGAG
GACCATCCTATCAGAAGATCGGACTCTGCCGTGTCAATTGTACATTTGAAACGTGCGCCCTTC
AAGGTTACAGTGATTGGTTCTGGTAACTGGGGGACCACCATCGCCAAAGTCATTGCGGAAAAC
ACAGAATTGCATTCCCATATCTTCGAGCCAGAGGTGAGAATGTGGGTTTTTGATGAAAAGATC
GGCGACGAAAATCTGACGGATATCATAAATACAAGACACCAGAACGTTAAATATCTACCCAAT
ATTGACCTGCCCCATAATCTAGTGGCCGATCCTGATCTTTTACACTCCATCAAGGGTGCTGAC
ATCCTTGTTTTCAACATCCCTCATCAATTTTTACCAAACATAGTCAAACAATTGCAAGGCCAC
GTGGCCCCTCATGTAAGGGCCATCTCGTGTCTAAAAGGGTTCGAGTTGGGCTCCAAGGGTGTG
CAATTGCTATCCTCCTATGTTACTGATGAGTTAGGAATCCAATGTGGCGCACTATCTGGTGCA
AACTTGGCACCGGAAGTGGCCAAGGAGCATTGGTCCGAAACCACCGTGGCTTACCAACTACCA
AAGGATTATCAAGGTGATGGCAAGGATGTAGATCATAAGATTTTGAAATTGCTGTTCCACAGA
CCTTACTTCCACGTCAATGTCATCGATGATGTTGCTGGTATATCCATTGCCGGTGCCTTGAAG
AACGTCGTGGCACTTGCATGTGGTTTCGTAGAAGGTATGGGATGGGGTAACAATGCCTCCGCA
GCCATTCAAAGGCTGGGTTTAGGTGAAATTATCAAGTTCGGTAGAATGTTTTTCCCAGAATCC
AAAGTCGAGACCTACTATCAAGAATCCGCTGGTGTTGCAGATCTGATCACCACCTGCTCAGGC
GGTAGAAACGTCAAGGTTGCCACATACATGGCCAAGACCGGTAAGTCAGCCTTGGAAGCAGAA
AAGGAATTGCTTAACGGTCAATCCGCCCAAGGGATAATCACATGCAGAGAAGTTCACGAGTGG
CTACAAACATGTGAGTTGACCCAAGAATTCCCATTATTCGAGGCAGTCTACCAGATAGTCTAC
AACAACGTCCGCATGGAAGACCTACCGGAGATGATTGAAGAGCTAGACATCGATGACGAATAG
SEQ ID NO 26: S. cerevisiae GPP1
ATGCCTTTGACCACAAAACCTTTATCTTTGAAAATCAACGCCGCTCTATTCGATGTTGACGGT
ACCATCATCATCTCTCAACCAGCCATTGCTGCTTTCTGGAGAGATTTCGGTAAAGACAAGCCT
TACTTCGATGCCGAACACGTTATTCACATCTCTCACGGTTGGAGAACTTACGATGCCATTGCC
AAGTTCGCTCCAGACTTTGCTGATGAAGAATACGTTAACAAGCTAGAAGGTGAAATCCCAGAA
AAGTACGGTGAACACTCCATCGAAGTTCCAGGTGCTGTCAAGTTGTGTAATGCTTTGAACGCC
TTGCCAAAGGAAAAATGGGCTGTCGCCACCTCTGGTACCCGTGACATGGCCAAGAAATGGTTC
GACATTTTGAAGATCAAGAGACCAGAATACTTCATCACCGCCAATGATGTCAAGCAAGGTAAG
CCTCACCCAGAACCATACTTAAAGGGTAGAAACGGTTTGGGTTTCCCAATTAATGAACAAGAC
CCATCCAAATCTAAGGTTGTTGTCTTTGAAGACGCACCAGCTGGTATTGCTGCTGGTAAGGCT
GCTGGCTGTAAAATCGTTGGTATTGCTACCACTTTCGATTTGGACTTCTTGAAGGAAAAGGGT
TGTGACATCATTGTCAAGAACCACGAATCTATCAGAGTCGGTGAATACAACGCTGAAACCGAT
GAAGTCGAATTGATCTTTGATGACTACTTATACGCTAAGGATGACTTGTTGAAATGGTAA
S. cerevisiae GPP2
SEQ ID NO: 27
ATGGGATTGACTACTAAACCTCTATCTTTGAAAGTTAACGCCGCTTTGTTCGACGTCGACGGT
ACCATTATCATCTCTCAACCAGCCATTGCTGCATTCTGGAGGGATTTCGGTAAGGACAAACCT
TATTTCGATGCTGAACACGTTATCCAAGTCTCGCATGGTTGGAGAACGTTTGATGCCATTGCT
AAGTTCGCTCCAGACTTTGCCAATGAAGAGTATGTTAACAAATTAGAAGCTGAAATTCCGGTC
AAGTACGGTGAAAAATCCATTGAAGTCCCAGGTGCAGTTAAGCTGTGCAACGCTTTGAACGCT
CTACCAAAAGAGAAATGGGCTGTGGCAACTTCCGGTACCCGTGATATGGCACAAAAATGGTTC
GAGCATCTGGGAATCAGGAGACCAAAGTACTTCATTACCGCTAATGATGTCAAACAGGGTAAG
CCTCATCCAGAACCATATCTGAAGGGCAGGAATGGCTTAGGATATCCGATCAATGAGCAAGAC
CCTTCCAAATCTAAGGTAGTAGTATTTGAAGACGCTCCAGCAGGTATTGCCGCCGGAAAAGCC
GCCGGTTGTAAGATCATTGGTATTGCCACTACTTTCGACTTGGACTTCCTAAAGGAAAAAGGC
TGTGACATCATTGTCAAAAACCACGAATCCATCAGAGTTGGCGGCTACAATGCCGAAACAGAC
GAAGTTGAATTCATTTTTGACGACTACTTATATGCTAAGGACGATCTGTTGAAATGGTAA
dmpF ORF synthetic codon optimized for Saccharomyces cerevisiae.
Reference sequence:
Pseudomonas sp. CF600
EMBL Accession number: X60835.1
SEQ ID NO: 28
ATGAATCAGAAACTGAAAGTAGCTATCATAGGTTCCGGTAATATCGGAACAGACCTGATGATT
AAGGTACTGCGTAATGCAAAGTACTTAGAAATGGGCGCGATGGTCGGTATCGATGCAGCCTCT
GATGGACTGGCCAGAGCTCAAAGAATGGGCGTTACGACTACTTATGCAGGTGTAGAAGGGCTA
ATCAAGCTTCCTGAATTTGCAGACATAGATTTCGTCTTCGATGCTACATCTGCATCAGCCCAC
GTTCAGAACGAGGCTTTATTAAGACAAGCTAAACCTGGTATTAGATTGATCGACCTTACCCCG
GCGGCAATCGGTCCTTACTGTGTCCCCGTAGTTAATCTCGAGGAACATTTGGGCAAGTTGAAC
GTTAACATGGTTACTTGCGGTGGCCAAGCTACTATTCCGATGGTCGCAGCTGTCTCACGTGTA
GCCAAAGTCCATTATGCTGAGATTGTTGCTTCTATTTCAAGCAAGAGTGCCGGACCTGGAACC
AGAGCCAATATAGATGAATTCACTGAGACAACCAGTAAAGCCATAGAAGTTATTGGTGGTGCT
GCAAAGGGTAAAGCTATAATTATTATGAACCCAGCTGAACCACCATTGATTATGAGGGATACG
GTGTATGTGCTTTCCGCCGCCGCTGATCAAGCCGCTGTCGCAGCTTCTGTGGCTGAAATGGTT
CAAGCGGTTCAAGCATACGTGCCAGGCTATAGGTTAAAACAACAGGTTCAGTTTGACGTGATT
CCCGAGTCCGCGCCACTAAACATCCCCGGTTTGGGGAGATTCAGCGGGTTGAAAACAAGTGTG
TTCCTAGAAGTAGAAGGTGCTGCTCATTATTTGCCAGCATACGCAGGAAACTTAGATATTATG
ACTTCCGCAGCGTTAGCTACAGCCGAACGTATGGCGCAATCAATGTTGAATGCATAG
dmpF ORF
SEQ ID NO: 29
MNQKLKVAIIGSGNIGTDLMIKVLRNAKYLEMGAMVGIDAASDGLARAQRMGVTTTYAGVEGL
IKLPEFADIDFVFDATSASAHVQNEALLRQAKPGIRLIDLTPAAIGPYCVPVVNLEEHLGKLN
VNMVTCGGQATIPMVAAVSRVAKVHYAEIVASISSKSAGPGTRANIDEFTETTSKAIEVIGGA
AKGKAIIIMNPAEPPLIMRDTVYVLSAAADQAAVAASVAEMVQAVQAYVPGYRLKQQVQFDVI
PESAPLNIPGLGRFSGLKTSVFLEVEGAAHYLPAYAGNLDIMTSAALATAERMAQSMLNA
primer dmpF-FW
SEQ ID NO: 30
CATTGATTGCGCCATACG
primer dmpF-RV
SEQ ID NO: 31
CCGGTAATATCGGAACAGAC
Wild type sequence of dmpF gene from Pseudomonas sp. CF600.
EMBL Accession number: X60835.1
SEQ ID NO: 32
ATGAACCAGAAACTCAAAGTCGCGATCATCGGTTCGGGCAATATCGGCACCGACCTGATGATC
AAGGTGCTGCGCAACGCCAAGTACCTGGAAATGGGCGCCATGGTCGGCATCGACGCCGCCTCC
GACGGCCTGGCCCGCGCCCAGCGCATGGGCGTGACGACCACCTATGCCGGCGTCGAAGGGCTG
ATCAAGCTGCCCGAATTCGCCGACATCGATTTCGTCTTCGACGCCACCTCGGCCAGTGCCCAC
GTGCAGAACGAGGCGCTGCTGCGCCAGGCCAAACCTGGCATCCGCCTGATCGACCTGACCCCG
GCGGCCATCGGCCCGTACTGCGTGCCGGTGGTCAATCTGGAGGAGCACCTCGGCAAGCTCAAC
GTCAACATGGTTACCTGCGGCGGCCAGGCGACCATCCCGATGGTCGCCGCGGTCTCCCGTGTG
GCCAAGGTCCATTACGCCGAGATCGTCGCCTCGATCAGCAGCAAGTCGGCCGGACCCGGCACC
CGCGCCAACATCGACGAGTTCACCGAGACCACCAGCAAAGCCATCGAAGTGATCGGTGGTGCG
GCCAAGGGCAAGGCGATCATCATCATGAACCCGGCTGAGCCGCCGCTGATCATGCGCGACACC
GTGTATGTGCTGTCCGCCGCCGCCGATCAGGCCGCCGTCGCGGCCTCGGTGGCGGAAATGGTT
CAGGCGGTGCAGGCCTACGTGCCCGGCTATCGCCTGAAGCAGCAGGTGCAGTTCGACGTGATC
CCCGAGTCCGCGCCGCTGAACATCCCCGGTCTCGGCCGGTTCAGCGGGTTGAAGACCTCGGTG
TTCCTCGAAGTCGAAGGCGCCGCCCATTACCTGCCGGCCTACGCCGGCAACCTCGACATCATG
ACCTCCGCCGCGCTGGCTACCGCCGAGCGTATGGCGCAGTCGATGTTGAACGCCTGA

What is claimed is:

1. Transgenic yeast cells comprising one or more recombinant heterologous, nucleic acid sequences encoding an NAD⁺-dependent acetylating acetaldehyde dehydrogenase (EC 1.2.1.10),
wherein said cells lack enzymatic activity needed for the NADH-dependent glycerol synthesis, or said cells have a reduced enzymatic activity with respect to the NADH-dependent glycerol synthesis compared to a corresponding wild-type yeast cell.

2. The cells of claim 1, wherein at least one said NAD⁺-dependent acetylating acetaldehyde dehydrogenase is represented by SEQ ID NO:2 or by SEQ ID:29 or a functional homolog of SEQ ID NO:2 or SEQ ID NO:29, said homolog having a sequence identity of at least 60% with SEQ ID NO:2 or SEQ ID NO:29.

3. The cells of claim 2, wherein at least one nucleic acid sequence encoding said dehydrogenase comprises a sequence according to SEQ ID NO:1, SEQ ID NO:28 or SEQ ID NO:32 or a functional homolog of SEQ ID NO:1, SEQ ID NO:28 or SEQ ID NO:32, said homolog having a sequence identity of at least 60% with SEQ ID NO:1.

4. The cells of claim 1, wherein said cells are free of NAD-dependent glycerol 3-phosphate dehydrogenase activity or have reduced NAD-dependent glycerol 3-phosphate dehydrogenase activity compared to corresponding wild-type cells, and/or
wherein the cells are either free of glycerol phosphate phosphatase activity or have reduced glycerol phosphate phosphatase activity compared to corresponding wild-type cells.

5. The cells of claim 4, which comprise a genomic mutation in at least one gene selected from the group consisting of GPD1, GPD2, GPP1 and GPP2.

6. The cells of claim 4, which are free of genes encoding NAD⁺-dependent glycerol 3-phosphate dehydrogenases (EC 1.1.1.8).

7. The cells of claim 1, further comprising one or more nucleic acid sequences encoding an acetyl-Coenzyme A synthetase activity (EC 6.2.1.1) and one or more nucleic acid sequences encoding NAD⁺-dependent alcohol dehydrogenase activity (EC 1.1.1.1).

8. The cells of claim 1 which are Saccharomycesceae, Kluyveromyces, Pichia, Zygosaccharomyces, or Brettanomyces.

9. The cells of claim 8 which are the S. cerevisiae strain deposited on 16 Jul. 2009 at the Centraal Bureau voor Schimmelcultures having deposit number CBS125049.

10. A vector for the functional expression of a nucleotide sequence encoding a polypeptide having enzymatic activity for converting acetyl-Coenzyme A into acetaldehyde in the cytosol of said yeast cell comprising said sequence operably linked to a promoter functional in the yeast cell,
wherein said polypeptide comprises a sequence according to SEQ ID NO:2, SEQ ID NO:29, or a functional homolog of said sequences.

11. A method to prepare ethanol from acetate and a fermentable carbohydrate which comprises culturing the yeast cells of claim 1 under anaerobic conditions.

12. The method of claim 11, wherein said culturing is carried out in a fermentation medium comprising acetate and carbohydrate in a molar ratio is 0.7 or less.

13. The method of claim 12, wherein at least part of the carbohydrate and at least part of the acetate has been obtained by hydrolysing a polysaccharide selected from the group of lignocelluloses, celluloses, hemicelluloses, and pectins.

14. The method of claim 13, wherein lignocellulosic biomass has been hydrolysed thereby obtaining the fermentable carbohydrate and acetate.

15. The cells of claim 5, wherein at least one said mutation is a complete deletion of said gene in comparison to the corresponding wild-type yeast gene.
